(19) United States
(12) Patent Application Publication
(10) Pub. No.: US 2025/0213564 A1
(43) Pub. Date: Jul. 3, 2025

(54) COMPOSITIONS AND METHODS FOR THE TREATMENT OF ALZHEIMER'S DISEASE AND OTHER NEUROGENERATIVE DISEASE

(71) Applicant: THE TRUSTEES OF COLUMBIA UNIVERSITY IN THE CITY OF NEW YORK, New York, NY (US)

(72) Inventors: Christine Ann DENNY, New York, NY (US); Briana K. CHEN, New York, NY (US); Holly HUNSBERGER, New York, NY (US)

(21) Appl. No.: 19/052,085

(22) Filed: Feb. 12, 2025

Related U.S. Application Data
(63) Continuation of application No. PCT/US2023/031141, filed on Aug. 25, 2023.
(60) Provisional application No. 63/400,910, filed on Aug. 25, 2022.

(51) Int. Cl. A61K 31/513; A61K 9/00; A61K 31/135; A61K 31/401; A61K 31/42; A61K 31/4245; A61K 31/4525; A61K 31/453; A61K 31/454; A61K 45/06; A61P 25/28

(52) U.S. Cl. CPC A61K 31/513; A61K 9/0019; A61K 31/135; A61K 31/401; A61K 31/42; A61K 31/4245; A61K 31/4525; A61K 31/453; A61K 31/454; A61K 45/06; A61P 25/28

(57) ABSTRACT

Methods and compositions that treat Alzheimer's disease and other neurodegenerative diseases and/or to ameliorate or improve symptoms associated with Alzheimer's disease. In some aspects, the compositions and methods use a serotonin 4 receptor (5-hydroxytryptamine (serotonin) receptor 4, or 5-HT₄R) agonist in combination with: (R,S)-ketamine, a (R,S)-ketamine analog, or a pharmaceutically acceptable salt, derivative, or metabolite thereof; an antagonist of the glutamate N-methyl-D-aspartate (NMDA) receptor (NMDAR); or an agonist of the α-amino-3-hydroxy-5-methyl-4-isoxazolepropionic acid (AMPA) receptor (AMPAR).

### CROSS-REFERENCE TO RELATED APPLICATIONS

The application is a Continuation of International Application No. PCT/US2023/031141, filed on Aug. 25, 2023, which claims priority to U.S. Provisional Application No. 63/400,910, filed Aug. 25, 2022, the disclosures of all of which are herein incorporated by reference in their entirety.

### TECHNICAL FIELD OF THE INVENTION

The present disclosure relates to the technical field of neurodegenerative diseases.

### BACKGROUND OF THE INVENTION

While Alzheimer's disease (AD), a neurodegenerative disorder leading to cognitive decline, was discovered 110 years ago, there are only a handful of approved drugs for symptomatic treatment. As of July 2020, the NIA supports 233 active clinical trials; however, many clinical trials of single drug administration have failed to meet endpoints. The complex pathophysiology of AD may necessitate combinatorial treatments rather than a single drug therapy.

Neuropsychiatric symptoms (NPS), such as depression and anxiety, have become a focus of numerous studies as early biomarkers and treatment targets for AD. However, there are currently only 9 clinical trials targeting NPS in AD. One of the biggest breakthroughs in the study of depression has been the discovery of (R,S)-ketamine as a rapid-acting antidepressant. (R,S)-ketamine is an N-Methyl-D-aspartic acid receptor (NDMAR) antagonist initially developed as an anesthetic, and has antidepressant effects that work as rapidly as 2 hours after administration, and has effects lasting for days to weeks after a single administration. Researchers have discussed using (R,S)-ketamine as a potential treatment for AD, but to date there is no registered clinical trial administering (R,S)-ketamine to AD patients.

In addition to NMDARs, serotonin type 4 receptors (5-HT₄Rs) have become of interest in modulating AD pathology. 5-HT₄R activation stimulates α-cleavage of the amyloid precursor protein (APP), leading to the release of soluble and neurotrophic sAPPα fragments. Activation of 5-HT₄Rs increases neuronal firing, increases neurogenesis, and can protect against as well as treat depression and anxiety. Numerous studies have shown that 5-HT₄R agonists are nootropic due to their ability to enhance learning and memory in rodents. However, there is a gap in knowledge in determining if combined drug administration targeting NMDARs and 5-HT₄Rs would have synergistic effects on both mood and cognition in AD.

### BRIEF SUMMARY OF THE INVENTION

Disclosed herein are compositions and methods which prevent or treat Alzheimer's disease (AD), including neuropsychiatric and cognitive defects.

Disclosed herein are studies to show the possible synergistic effects of (R,S)-ketamine and prucalopride, a 5-HT₄R agonist, in a mouse model of AD. Mice were administered at varying doses either: 1) saline; 2) (R,S)-ketamine; 3) prucalopride; or 4) (R,S)-ketamine and prucalopride to simultaneously target co-morbid neuropsychiatric and cognitive deficits in Control (Ctrl) or APP/PS1 (AD) mice. Assays were then administered to measure cognition, perseverative behavior, hyponeophagia, and sleep. Combined (R,S)-ketamine and prucalopride administration decreased sleep amplitude in AD mice. Combined (R,S)-ketamine and prucalopride administration, but not single drug administration, decreased perseverative behavior in Ctrl mice. Most notably, combined (R,S)-ketamine and prucalopride administration improved cognitive decline in AD mice. These results indicated that combined administration of (R,S)-ketamine and prucalopride is a novel multimodal therapeutic strategy to treat AD.

The current compositions and methods can be used to develop a novel compound with greater and longer-lasting efficacy for treating AD or other neurodegenerative diseases and/or to ameliorate or improve symptoms associated with AD.

In one aspect, the present disclosure provides for a method for treating AD or another neurodegenerative disease in a subject in need thereof. The method comprises administering an effective amount of one or more compositions comprising an agonist or activator of serotonin 4 receptor (5-HT₄R) (e.g., an agonist of serotonin 4 receptor (5-HT₄R)), or a pharmaceutically acceptable salt, analog, derivative, or metabolite thereof, and (R,S)-ketamine, a (R,S)-ketamine analog, or a pharmaceutically acceptable salt, derivative, or metabolite thereof.

The present disclosure further provides for a method for treating AD or another neurodegenerative disease in a subject in need thereof by administering an effective amount of one or more compositions comprising an agonist or activator of serotonin 4 receptor (5-HT₄R) (e.g., an agonist of serotonin 4 receptor (5-HT₄R)), or a pharmaceutically acceptable salt, analog, derivative, or metabolite thereof, and an antagonist of the glutamate N-methyl-D-aspartate (NMDA) receptor (NMDAR).

Additionally, the present disclosure provides for a method for treating AD or another neurodegenerative disease in a subject in need thereof by administering an effective amount of one or more compositions comprising an agonist or activator of serotonin 4 receptor (5-HT₄R) (e.g., an agonist of serotonin 4 receptor (5-HT₄R)), or a pharmaceutically acceptable salt, analog, derivative, or metabolite thereof, and an agonist of the α-amino-3-hydroxy-5-methyl-4-isoxazolepropionic acid (AMPA) receptor (AMPAR).

In one aspect, the present disclosure provides for a method for improving or ameliorating symptoms associated with AD in a subject in need thereof. The method comprises administering an effective amount of one or more compositions comprising an agonist or activator of serotonin 4 receptor (5-HT₄R) (e.g., an agonist of serotonin 4 receptor (5-HT₄R)), or a pharmaceutically acceptable salt, analog, derivative, or metabolite thereof, and (R,S)-ketamine, a (R,S)-ketamine analog, or a pharmaceutically acceptable salt, derivative, or metabolite thereof.

The present disclosure further provides for a method for improving or ameliorating symptoms associated with AD in a subject in need thereof by administering an effective amount of one or more compositions comprising an agonist or activator of serotonin 4 receptor (5-HT₄R) (e.g., an agonist of serotonin 4 receptor (5-HT₄R)), or a pharmaceutically acceptable salt, analog, derivative, or metabolite thereof, and an antagonist of the glutamate N-methyl-D-aspartate (NMDA) receptor (NMDAR).

Additionally, the present disclosure provides for a method for improving or ameliorating symptoms associated with AD in a subject in need thereof by administering an effective amount of one or more compositions comprising an agonist or activator of serotonin 4 receptor (5-HT₄R) (e.g., an agonist of serotonin 4 receptor (5-HT₄R)), or a pharmaceutically acceptable salt, analog, derivative, or metabolite thereof, and an agonist of the α-amino-3-hydroxy-5-methyl-4-isoxazolepropionic acid (AMPA) receptor (AMPAR).

In some embodiments, the activator or agonist of 5-HT₄R and the (R,S)-ketamine, a (R,S)-ketamine analog, or a pharmaceutically acceptable salt, derivative, or metabolite thereof, the NMDAR antagonist, or the AMPAR agonist are in the same composition. In some embodiments, the agonist or activator of 5-HT₄R and the (R,S)-ketamine, a (R,S)-ketamine analog, or a pharmaceutically acceptable salt, derivative, or metabolite thereof, the NMDAR antagonist, or the AMPAR agonist are in different compositions. In these embodiments, the compositions can be administered simultaneously or sequentially.

The activator or agonist of 5-HT₄R includes but is not limited to 1-(4-amino-5-chloro-2-methoxyphenyl)-3-[1(n-butyl)-4-piperidinyl]-1-propanone HCl (RS-67,333 or RS67333), 4-amino-5-chloro-2,3-dihydro-N-[1-3-methoxypropyl)-4-piperidinyl]-7-benzofuran carboxamide monohydrochloride (prucalopride), 4-[4-[4-tetrahydrofuran-3-yloxy)-benzo[d]isoxazol-3-yloxymethyl]-piperidin-1-ylmethyl]-tetrahydropyran-4-ol (PF-04995274), or combinations thereof.

In some embodiments, the disclosure provides for a method for treating AD or another neurodegenerative disease in a subject in need thereof by administering an effective amount of one or more compositions comprising prucalopride or a pharmaceutically acceptable salt, analog, derivative, or metabolite thereof, and (R,S)-ketamine, a (R,S)-kctaminc analog, or a pharmaceutically acceptable salt, derivative, or metabolite thereof.

In some embodiments, the disclosure provides for a method for improving or ameliorating symptoms associated with AD in a subject in need thereof by administering an effective amount of one or more compositions comprising prucalopride or a pharmaceutically acceptable salt, analog, derivative, or metabolite thereof, and (R,S)-ketamine, a (R,S)-ketamine analog, or a pharmaceutically acceptable salt, derivative, or metabolite thereof.

In some embodiments, the prucalopride and the (R,S)-ketamine, a (R,S)-ketamine analog, or a pharmaceutically acceptable salt, derivative, or metabolite thereof are in the same composition. In some embodiments, the prucalopride and the (R,S)-ketamine, a (R,S)-ketamine analog, or a pharmaceutically acceptable salt, derivative, or metabolite thereof are in different compositions. In these embodiments, the compositions can be administered simultaneously or sequentially.

In some embodiments, other neurodegenerative diseases include but are not limited to Parkinson's disease (PD), Amyotrophic Lateral Sclerosis (ALS), Frontal-Temporal Degeneration, Huntington's disease, multiple sclerosis, and dementia.

In some embodiments, the symptoms associated with AD are neuropsychiatric symptoms, including but not limited to anxiety, depression, sleep disorders, appetite disorders, apathy, psychosis, perseverative behavior, agitation, aggression/irritability, euphoria, rumination, and combinations thereof.

In some embodiments, the symptoms associated with AD are cognitive including loss of memory, impairments of language and visuospatial function, impaired learning, delusions/hallucinations, disinhibition, overall impairments in executive functions, and combinations thereof.

In some embodiments, the symptoms are both neuropsychiatric and cognitive.

In certain embodiments, the composition or compositions can be administered by a variety of means including but not limited to oral, intravenous (i.v. or IV), intranasal (i.n. or IN), intramuscular (i.m. or IM), caudal, intrathecal, and subcutaneous (s.c.) routes.

The subject may be a mammal. In certain embodiments, the subject is a human. The subject may be female or male.

### BRIEF DESCRIPTION OF THE SEVERAL VIEWS OF THE DRAWINGS

For the purpose of illustrating the invention, there are depicted in drawings certain embodiments of the invention. However, the invention is not limited to the precise arrangements and instrumentalities of the embodiments depicted in the drawings.

FIGS. 1A-1H show that combined prophylactic (R,S)-ketamine and prucalopride administration decreased sleep amplitude in male APP/PS1 mice. FIG. 1A shows the experimental design for the result in FIGS. 1-3. FIG. 1B is a graph of sleep activity of control mice (Ctrl) administered saline and those administered (R,S)-ketamine+prucalopride (10+3 mg/kg). FIG. 1C is a graph of sleep activity of AD mice administered saline and those administered (R,S)-ketamine+prucalopride (10+3 mg/kg). Mice exhibited increased activity during the night. However, activity was decreased significantly in the combined (R,S)-ketamine and prucalopride-treated Ctrl and AD mice. FIG. 1D shows the sleep amplitude in each group of mice. Amplitude was decreased in (R,S)-ketamine and prucalopride-treated AD mice compared to (R,S)-ketamine and prucalopride-treated Ctrl mice. FIG. 1E show the overall time sleeping in each group of mice. There was an overall Time×Genotype×Drug interaction across 4 days where the combination drug improved sleep in the AD mice. FIG. 1F shows the average sleep percentage of each group of mice. Average sleep percentage was similar among the groups. FIG. 1G shows the percentage of sleep during light phase for each group of mice. During the light phase, AD mice exhibited less overall sleep compared to Ctrl mice. Drug administration did not impact this phenotype. FIG. 1H shows the percentage of sleep during the dark phase for each group of mice. There was no effect of Drug or Genotype during the dark phase. (n=4-6 mice per group). Error bars represent ±SEM. *p<0.05. **p<0.01. ***p<0.0001. Sal, saline; K, (R,S)-ketamine; P, prucalopride; CFC, contextual fear conditioning; NSF, novelty suppressed feeding; MB, marble burying.

FIGS. 2A-2P show that combined (R,S)-ketamine and prucalopride administration decreased perseverative behavior and improves cognition in male APP/PSI mice. Mice were given a single injection of drug or combined injection and then tested in a number of behavioral assays starting approximately 5 days later (FIG. 1A). FIG. 2A shows the graph of the fraction of Ctrl mice treatment groups not feeding versus latency to feed in OF in seconds in the NSF paradigm. FIG. 2B shows the graph of the fraction of AD mice treatment groups not feeding versus latency to feed in OF in seconds in the NSF paradigm. FIG. 2C is a graph of the latency to feed in OF in seconds of all groups of mice in the NSF paradigm. FIGS. 2A-2C show in the NSF paradigm, there were no differences in the latency to approach the food pellet between the groups. FIG. 2D shows the graph of the fraction of Ctrl mice treatment groups not feeding versus latency to feed in HC in seconds. FIG. 2E shows the graph of the fraction of AD mice treatment groups not feeding versus latency to feed in HC in seconds. FIG. 2F is a graph of the latency to feed in HC in seconds of all groups of mice. FIGS. 2D-2F show Ctrl mice treated with prucalopride approached the pellet more quickly when placed in their home cage when compared to Ctrl mice. FIG. 2G shows the weight of each group of mice across time. FIG. 2H shows the weight difference in the groups of mice. FIGS. 2G and 2H shows that AD mice weighed more than Ctrl mice, but neither weight nor weight loss was impacted by the administration of prucalopride. FIG. 2I shows the food consumed in the home cage was similar among the groups. FIG. 2J shows the number of marbles buried in the MB task for each group of mice, and shows combined (R,S)-ketamine and prucalopride administration, but not single drug administration, decreased perseverative behavior in Ctrl mice. In AD mice, all drugs reduced perseverative behavior. FIG. 2K shows freezing frequency during training in Ctrl mice treatment groups. (R,S)-ketamine-treated Ctrl mice treated exhibited increased freezing during training when compared to other groups. FIG. 2L shows freezing frequency during training in AD mice showing that drug administration did not impact learning in AD mice. FIG. 2M shows freezing frequency in both Ctrl and AD mice. Neither (R,S)-ketamine nor prucalopride, whether administered solely or jointly, affected learning in AD mice. FIG. 2N is a graph showing freezing frequency during re-exposure in Ctrl mice, showing that during memory retrieval, all groups of Ctrl mice froze comparably. FIG. 2O is a graph showing freezing during re-exposure in AD mice. FIG. 2P shows freezing during re-exposure in all groups of mice. FIGS. 2O and 2P show that saline-injected AD mice were impaired in memory retrieval (i.e., decreased freezing) when compared to Ctrl mice. Notably, only combined (R,S)-ketamine and prucalopride administration increased freezing behavior, a proxy for memory retrieval, in AD mice. (n=4-6 mice per group). Error bars represent ±SEM. *p<0.05. **p<0.01. ***p<0.0001. Sal, saline; K, (R,S)-ketamine; P, prucalopride; CFC, contextual fear conditioning; NSF, novelty suppressed feeding; MB, marble burying.

FIG. 3 illustrates that the combined prophylactic administration of (R,S)-ketamine and prucalopride administration decreases perseverative behavior in female APP/PS1 mice. Based on the number of marbles buried in the MB task for each group of mice, (R,S)-ketamine and combined (R,S)-ketamine and prucalopride administration decreased perseverative behavior in Ctrl mice. In AD mice, combined (R,S)-ketamine and prucalopride decreased perseverative behavior. Error bars represent ±SEM. *p<0.05. **p<0.01. ***p<0.0001.

FIGS. 4A-4G demonstrate that chronic combined (R,S)-ketamine and prucalopride administration improves memory retrieval in 2-month-old male APP/PS1 mice. FIG. 4A summarizes the experimental design. FIG. 4B shows that saline-treated AD mice were impaired during memory encoding when compared with saline-treated Ctrl mice. FIG. 4C shows that in AD mice, 1×(R,S)-ketamine (30 mg/kg), 7×prucalopride (3 mg/kg), and 1×(R,S)-ketamine (10 mg/kg)+7×prucalopride (3 mg/kg) improved memory retrieval. FIGS. 4D and 4E show that all groups exhibited comparable immobility time in days 1 and 2 of the forced swim test (FST). FIG. 4F shows that all groups exhibited a comparable weight loss over time.

FIGS. 5A-5G reveal that chronic combined (R,S)-ketamine and prucalopride administration decreases perseverative behavior in 2-month-old female APP/PS1 mice. FIG. 5A summarizes the experimental design. FIG. 5B shows that saline-treated AD mice were impaired during memory encoding when compared with saline-treated Ctrl mice. FIG. 5C shows that saline-treated AD mice were impaired during memory retrieval when compared with saline-treated Ctrl mice. FIGS. 5D and 5E show that all groups exhibited comparable immobility time in days 1 and 2 of the FST. FIG. 5F shows that in the MB task, all groups of Ctrl mice buried a comparable percentage of marbles. In AD mice, 7×prucalopride (1.5 and 3 mg/kg), and 1×(R,S)-ketamine (10 mg/kg)+7×prucalopride (3 mg/kg) decreased the percentage of marbles buried. FIG. 5G shows that all groups exhibited a comparable weight loss over time.

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

In addition, ranges provided in the specification and appended claims include both end points and all points between the end points. Therefore, a range of 1.0 to 2.0 includes 1.0, 2.0, and all points between 1.0 and 2.0.

Unless otherwise indicated, all numbers expressing quantities of ingredients, properties and so forth used in the present disclosure and associated claims are to be understood as being modified in all instances by the term “about.” Accordingly, unless indicated to the contrary, the numerical parameters set forth in this disclosure and attached claims are approximations that may vary depending upon the desired properties sought to be obtained by the examples of the present invention. The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “cither,” “one of,” “only one of,” or “exactly one of “consisting essentially of,” when used in the claims, shall have its ordinary meaning as used in the field of patent law.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of and “consisting essentially of shall be closed or semi-closed transitional phrases, respectively.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

Certain terms are discussed immediately hereinafter, or elsewhere in the specification, to provide additional guidance to the practitioner in describing the methods of the inventions described herein and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of any invention disclosed herein or any exemplified term. Likewise, the invention is not limited to its preferred embodiments.

The term “subject” as used in this application means mammals. Mammals include canines, felines, rodents, bovine, equines, porcines, ovines, and primates. Thus, the compositions and methods disclosed herein can be used in veterinary medicine, e.g., to treat companion animals, farm animals, laboratory animals in zoological parks, and animals in the wild. The disclosure is particularly desirable for human medical applications

The term “patient” as used in this application means a human subject. In some embodiments of the present disclosure, the patient has been diagnosed with Alzheimer's disease or another neurodegenerative disease or is suspected of having Alzheimer's disease or another neurodegenerative disease or is at risk for Alzheimer's disease or another neurodegenerative disease.

The phrase “therapeutically effective amount” or “effective amount” or “therapeutically effective dose” or “effective dose” is used herein to mean an amount sufficient to cause an improvement in a clinically significant condition in the subject, or delays or minimizes or mitigates one or more symptoms associated with the disease or disorder, or results in a desired beneficial change of physiology in the subject.

The terms “treat”, “treatment”, and the like refer to a means to slow down, relieve, ameliorate or alleviate at least one of the symptoms of the disease or disorder, or reverse the disease or disorder after its onset.

The terms “prevent”, “prevention”, and the like refer to acting prior to overt disease or disorder onset, to prevent the disease or disorder from developing or minimize the extent of the disease or disorder or slow its course of development.

The term “in need thereof” would be a subject who has been diagnosed with Alzheimer's disease or another neurodegenerative disease or is suspected of having Alzheimer's disease or another neurodegenerative disease or is at risk for Alzheimer's disease or another neurodegenerative disease.

The term “agent” as used herein means a substance that produces or is capable of producing an effect and would include, but is not limited to, chemicals, pharmaceuticals, biologics, small organic molecules, antibodies, nucleic acids, peptides, and proteins.

As used herein “an adverse effect” is an unwanted reaction caused by the administration of a drug.

The term “pharmaceutically acceptable derivative” refers to any pharmaceutically acceptable salt, solvate, prodrug, e.g., ester, or other precursors, of a compound which upon administration to the recipient is capable of providing (directly or indirectly) the active compound or an active metabolite or residue thereof. Such salts include pharmaceutically acceptable basic or acid addition salts as well as pharmaceutically acceptable metal salts, ammonium salts and alkylated ammonium salts. Such derivatives are recognizable to those skilled in the art, without undue experimentation. Derivatives are described, for example, in Burger's Medicinal Chemistry and Drug Discovery, 5th Edition, Vol 1: Principles and Practice, which is incorporated herein by reference. In certain embodiments, pharmaceutically acceptable derivatives include salts, solvates, esters, carbamates, and phosphate esters.

Abbreviations: Alzheimer's disease (AD); Control (Ctrl); contextual fear conditioning (CFC); novelty suppressed feeding (NSF); housed cage (HC); open field (OF); marble burying (MB); (R,S,)-ketamine (K); prucalopride (P).

The term “pharmaceutically acceptable derivative” refers to any pharmaceutically acceptable salt, solvate, prodrug, e.g., ester, or other precursors, of a compound which upon administration to the recipient is capable of providing (directly or indirectly) the active compound or an active metabolite or residue thereof. Such salts include pharmaceutically acceptable basic or acid addition salts as well as pharmaceutically acceptable metal salts, ammonium salts and alkylated ammonium salts. Such derivatives are recognizable to those skilled in the art, without undue experimentation. Derivatives are described, for example, in Burger's Medicinal Chemistry and Drug Discovery, 5th Edition, Vol 1: Principles and Practice, which is incorporated herein by reference. In certain embodiments, pharmaceutically acceptable derivatives include salts, solvates, esters, carbamates, and phosphate esters.

The Combined Administration of Two Different Agents or Compounds as a Treatment for Alzheimer's Disease and/or For the Amelioration or Improvement of Symptoms Associated with Alzheimer's Disease

The current disclosure employs a combined administration of two different compounds or agents, a 5-HT₄R agonist or activator, and ketamine, a ketamine analog, or a pharmaceutically acceptable salt. derivative, or metabolite thereof, or an NMDAR antagonist or an AMPAR agonist, as treatment for Alzheimer's disease or other neurodegenerative diseases and/or for the amelioration or improvement of symptoms associated with Alzheimer's disease or other neurodegenerative disease. At varying dosages, this combination successfully decreased sleep amplitude, perseverative behavior and cognitive decline by increasing memory retrieval in an AD mouse model. While the intervention of the behaviors can be accomplished administering these compounds or agents in one or more compositions, the disclosure herein also provides the basis for a novel compound that will integrate the functional structures of both of the compounds or agents. As such, this disclosure presents a pharmacological intervention for treatment for Alzheimer's disease or other neurodegenerative diseases and/or for the amelioration or improvement of symptoms associated with Alzheimer's disease or neurodegenerative disease.

Alzheimer's disease is the leading cause of dementia, however, there are only a few approved drugs for treatment. To address this, mice were administered at varying doses either: 1) saline; 2) (R,S)-ketamine; 3) prucalopride; or 4) (R,S)-ketamine and prucalopride to simultaneously target co-morbid neuropsychiatric and cognitive deficits in Control (Ctrl) or APP/PS1 (AD) mice. Assays were then administered to measure cognition, perseverative behavior, hyponeophagia, and sleep.

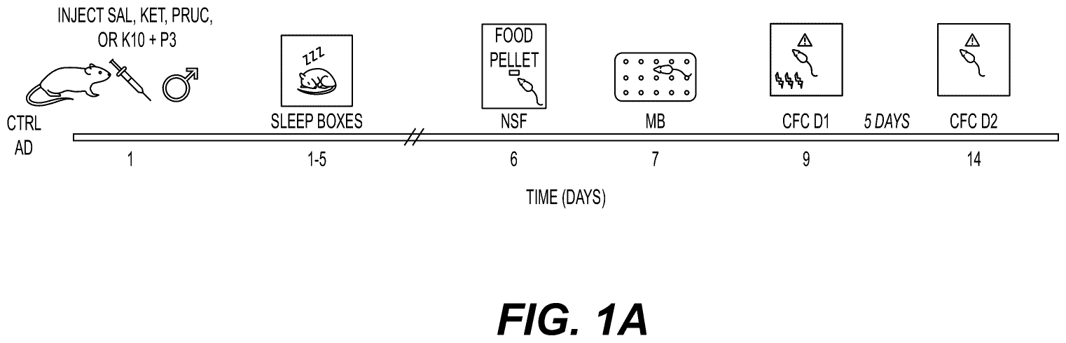

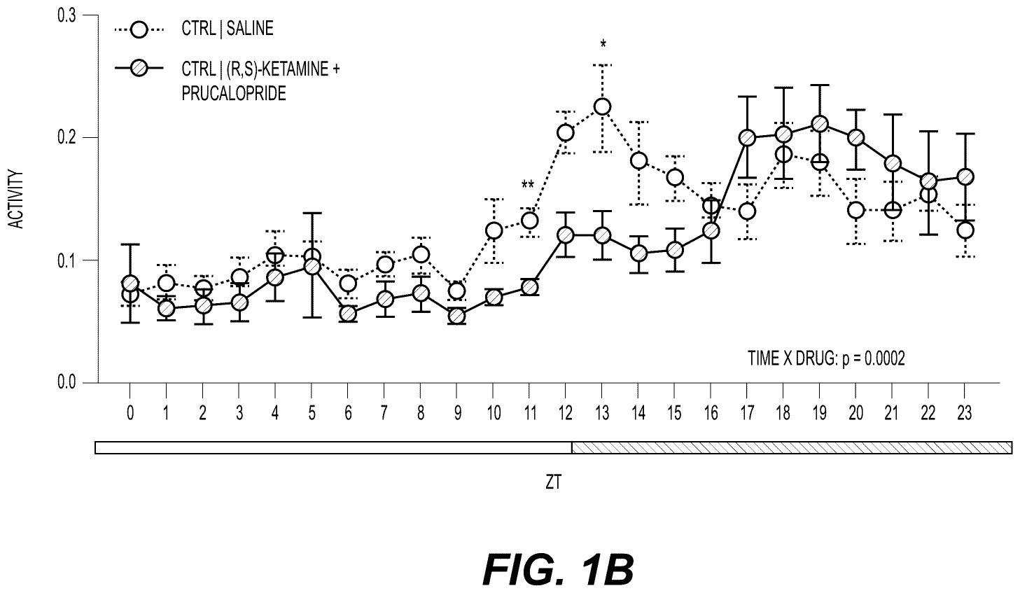

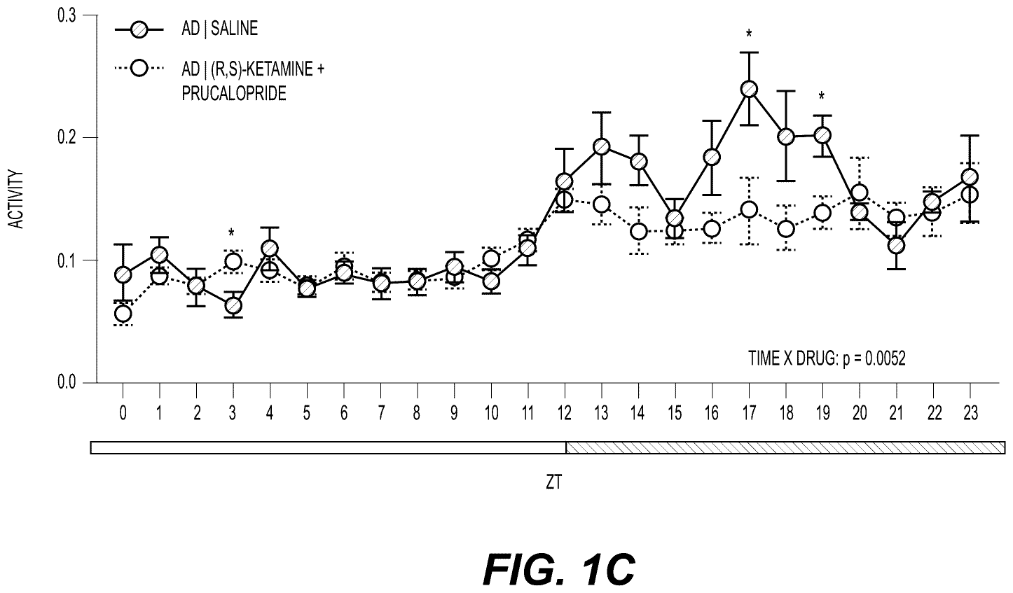

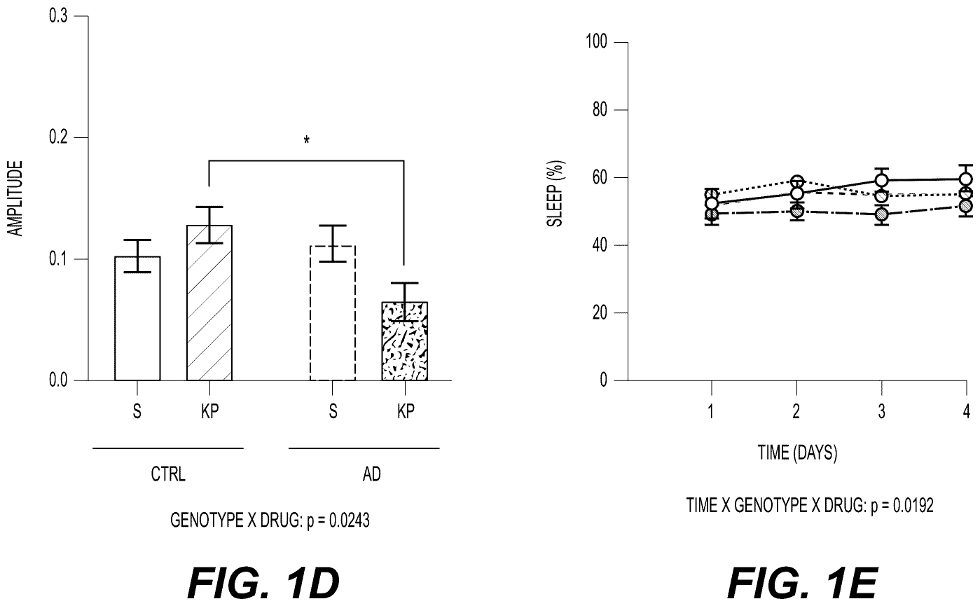

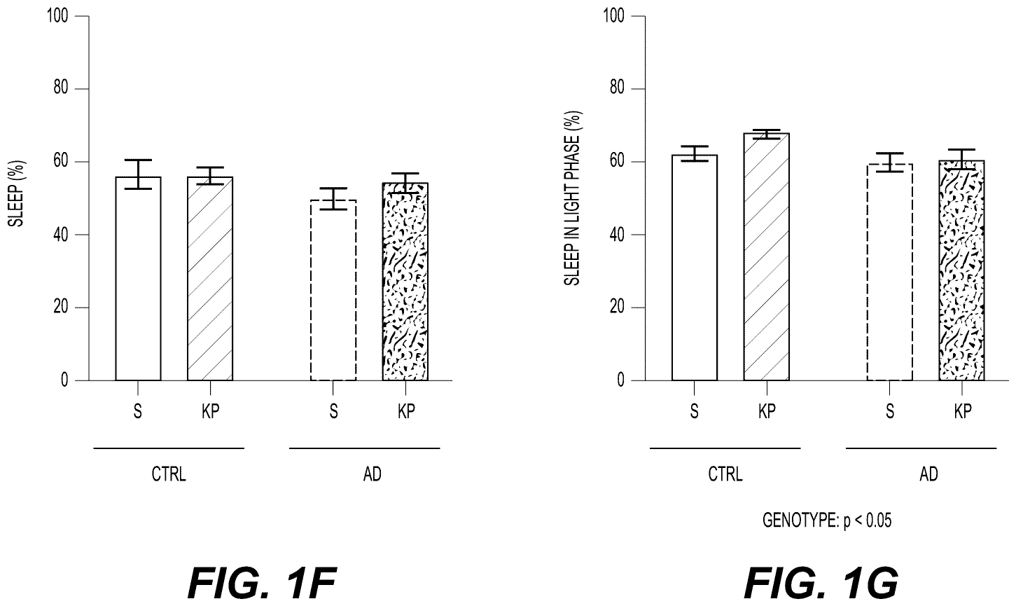

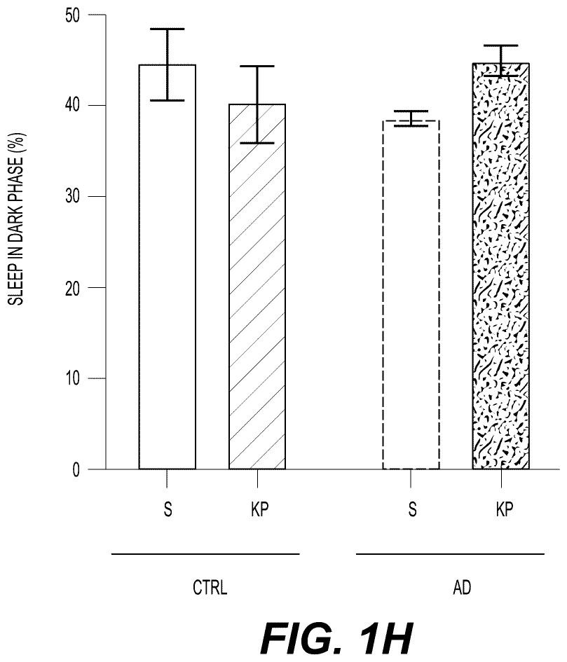

Specifically, a combined dose of prucalopride and (R,S)-ketamine, as well as doses of individual drug and saline were administered to Ctrl and a mouse model of Alzheimer's disease. Following drug administration, the mice were placed in Piezo sleep boxes for four days. Six days after drug administration, mice were assayed for hyponcophagia using novelty-suppressed feeding (NSF). Seven days after drug administration, mice were assayed for perseverative behavior in a marble burying assay (MB). Nine days after drug administration, mice were assayed for cognition and learning in a contextual fear conditioning (CFC) assay. See FIG. 1A.

Combined (R,S)-ketamine and prucalopride administration decreased sleep amplitude in AD mice. Combined (R,S)-ketamine and prucalopride administration, but not single drug administration, decreased perseverative behavior in Ctrl mice. Most notably, combined (R,S)-ketamine and prucalopride administration improved cognitive decline in AD mice. These results indicated that combined administration of (R,S)-ketamine and prucalopride is a novel multimodal therapeutic strategy to treat AD.

The present compounds, agents or compositions may be administered by various routes, including oral, intravenous (i.v. or IV), intranasal (i.n. or IN), intramuscular (i.m. or IM), caudal, intrathecal, and subcutaneous (s.c.) routes.

### 5-HT₄R Activators or Agonists

The serotonin 4 receptor (5-HT₄R) is a G-protein coupled receptor (GPCR) that activates G protein Gs and stimulates the cAMP/PKA signaling pathway, resulting in the phosphorylation of cAMP response element binding protein (CREB) and as a consequence the expression of a number of genes involved in neuroplasticity (Vidal et al., 2014). The majority of 5-HT₄Rs are expressed in the brain of primates and rodents specifically in the medium spiny neurons of the striatum, the ammon's horns (Cornu Ammonis 1 (CA1) and CA3) of the hippocampus, the granule cells of the dentate gyrus and glutamatergic neurons in the cortex and amygdala (Rebholz et al., 2018). In addition, 5-HT₄Rs are also found in hypothalamus, ventral pallidum, olfactory bulbs, septal area, and substantia nigra. Mice lacking the 5-HT₄R display anhedonia and a context-dependent anxiety-like behavior (Amigo et al., 2016) and various 5-HT₄R agonists can exert an antidepressant and anxiolytic-like activity (Samuels et al., 2016).

Whether in humans or in rodents, the expression of the 5-HT₄ is found in the limbic regions (mPFC, HPC and NAc). In addition, the basal ganglia, i.e., the caudate nucleus and the lenticular nucleus (putamen and pallidum), the black matter, and the amygdala, also express the 5-HT₄ receptor. The 5-HT₄ receptor is expressed at the somatodendritic level and at the level of the axon terminals of efferent spinal GABAergic neurons of the striatum, the CA1 and CA3 of the hippocampus, the granular cells of the dentate gyrus, and glutamatergic neurons of the cortex, the hippocampus and the amygdala.

5-HT₄ receptors are also found at the peripheral level, in particular at the cardiac level, where activation thereof exerts a positive inotropic effect, at the level of the gastro-intestinal tract where it is involved in intestinal motility, at the level of the adrenal glands where it plays a role in secretion of corticosterone, and at the level of the bladder where it causes contraction of the smooth muscles.

The 5-HT₄ receptor has seven transmembrane domains. The N-terminal region faces towards the extracellular environment, whereas the C-terminal domain, coupled to a Gs protein, faces towards the cytoplasm. The activation of the 5-HT₄ receptor, e.g., by an agonist, can lead to the recruitment of the Gs protein which stimulates adenylate cyclase (AC) which is responsible for the production of cAMP. Protein kinase A (PKA), activated by the CAMP, modulates different ionic currents and in particular potassium currents, the inhibition of which results in neuronal hyperexcitability. The PKA is also capable of phosphorylating the protein binding the response element to the cAMP (CREB-CAMP response element binding protein), which results in an increase in the transcription of neurotrophic brain factor (BDNF, brain-derived neurotrophic factor), involved in cognition, mood and cell survival.

In addition to NMDARs, 5-HT₄Rs have become of interest in treating AD pathology. 5-HT₄R activation stimulates a-cleavage of the amyloid precursor protein (APP), leading to the release of soluble and neurotrophic soluble APP (sAPPa) fragments (Cochet et al. 2013). Activation of 5-HT₄Rs increases neuronal firing (Schill et al. 2020), increases neurogenesis (Imoto et al. 2015) and can protect against depression and anxiety (Chen et al. 2020). Numerous other studies have shown that 5-HT₄R agonists are considered nootropics due to their ability to enhance learning and memory in rodents. For example, there is a current clinical trial investigating whether the 5-HT₄R agonist prucalopride (MOTEGRITY™) has effects on emotional processing, cognition, and neural activity in healthy volunteers (University of Oxford 2018). A prior study showed that prucalopride and donepezil, a cholinesterase inhibitor, showed synergistic effects in rescuing memory (Cachard-Chastel et al. 2008). Furthermore, a study showed that prucalopride and PRX-01340, both 5-HT₄R agonists, increased cortical ACh and histamine levels, and stimulated hippocampal q power, changes that are consistent with positive effects on cognitive processes (Johnson et al. 2012).

In summary, targeting 5-HT₄Rs does seem to be an interesting and novel target for improving memory loss. Although these examples give premise to our study, these treatments are not fully effective in treating AD, and therefore, new combinatorial approaches are needed (Cochet et al. 2013).

The term “agonist” may refer to a substance, an agent or a compound capable of binding to and activating one or more receptors, such as 5-HT₄R. The term “agonist” may refer to a compound having the ability to initiate or enhance a biological function of a target protein (e.g., one or more receptors, such as 5-HT₄R), whether by enhancing or initiating the activity or expression of the target protein. 5-HT₄R agonists may be compounds that activate the action of the 5-HT₄ receptor. The term “agonist” may be defined in the context of the biological role of the target protein. In one embodiment, an agonist is an agent that binds to a receptor (e.g., 5-HT₄R) and activates the receptor to produce a biological response. While agonists provided herein can specifically interact with (e.g., bind to) the target protein, compounds that initiate or enhance a biological activity of the target protein by interacting with other members of the signal transduction pathway of which the target protein is a member are also specifically included within this definition. A 5-HT₄R agonist may be a compound or an agent that activates the action of 5-HT₄R. A 5-HT₄R agonist may be any agent that acts directly or indirectly through or upon 5-HT₄R to produce a pharmacological effect. The terms “agonist of 5-HT₄R”, “agonist of the 5-HT₄ receptor”, “5-HT₄ receptor agonist”, and “5-HT₄R agonist” are used interchangeably herein.

The 5-HT₄R agonist may be selective for 5-HT₄ receptors, or it may be non-selective, exhibiting agonist or antagonist activity at other serotonin receptors. In one embodiment, the 5-HT₄R agonist is selective for 5-HT₄ receptors.

The 5-HT₄R agonists may include full agonists, partial agonists, or mixed 5-HT₄R agonists/antagonists.

“Full agonists” may refer to agents able to bind to and activate a receptor with the maximum response that an agonist can elicit at the receptor. An agent may act as a full agonist in some tissues and as a partial agonist in other tissues, depending upon the relative numbers of receptors and differences in receptor coupling.

“Partial agonists” may refer to compounds able to bind and activate a given receptor but having only partial efficacy at the receptor relative to a “full agonist” or complete agonist. Partial agonists can act as antagonists when competing with a full agonist for receptor occupancy and producing a net decrease in the receptor activation compared to the effects or activation observed with the full agonist alone. Partial agonists may refer to mixed agonists/antagonists, which differentially affect a receptor function within different dose ranges. For example, partial agonists may serve as agonists at lower doses, and as antagonists at higher doses. Partial agonists may be compounds that have reduced efficacy for inducing conformational change in receptors (typically 40-80%) relative to full agonists, and which may induce agonist effects at low dose but antagonist effects at high dose.

The 5-HT₄R agonist may be an indole, a benzamide, a benzoate, an arylketone or a benzamide.

Non-limiting examples of 5-HT₄R agonists include, 1-(4-amino-5-chloro-2-methoxyphenyl)-3-[1(n-butyl)-4-piperidinyl]-1-propanone HCl (RS-67,333 or RS67333), 4-amino-5-chloro-2,3-dihydro-N-[1-3-methoxypropyl)-4-piperidinyl]-7-benzofuran carboxamide monohydrochloride (prucalopride), 4-[4-[4-Tetrahydrofuran-3-yloxy)-benzo[d]isoxazol-3-yloxymethyl]-piperidin-1-ylmethyl]-tetrahydropyran-4-ol (PF-04995274), and combinations thereof.

Non-limiting examples of 5-HT₄R agonists also include, 2-[1-(4-Piperonyl)piperazinyl]benzothiazole (PPB), 5-methoxytryptamine, PRX-03140, cisapride ((±)-cis-4-amino-5-chloro-N-[1-[3-(4-fluorophenoxy)propyl]-3-methoxy-4-piperidinyl]-2-methoxybenzamide monohydrate), BIMU-8 (2,3-Dihydro-N-[(3-endo)-8-methyl-8-azabicyclo[3.2.1]oct-3-yl]-3-(1-methylethyl)-2-oxo-1H-benzimidazole-1-carboxamide, RS67506 (methylsulphonylamino)ethyl-4-piperidinyl]-1-propanone hydrochloride), mosapride (4-amino-5-chloro-2-ethoxy-N-[[4-[(4-fluorophenyl)methyl]-2-morpholinyl]methyl]benzamide citrate), tegascrod (2-[(5-Methoxy-1H-indol-3-yl)methylene]-N-pentyl-hydrazinecarboximidamide malcate), ML10302 (4-Amino-5-chloro-2-methoxybenzoic acid 2-(1-piperidinyl)ethyl ester hydrochloride), velusetrag (TD-5108) (N-[(1R,3R,5S)-8-[(2R)-2-hydroxy-3-(N-methylmethanesulfonamido)propyl]-8-azabicyclo[3.2.1]octan-3-yl]-2-oxo-1-(propan-2-yl)-1,2-dihydroquinoline-3-carboxamide), naropride (ATI-7505) ([(3R)-1-azabicyclo[2.2.2]octan-3-yl]6-[(3S,4R)-4-[(4-amino-5-chloro-2-methoxybenzoyl)amino]-3-methoxypiperidin-1-yl]hexanoate, cinitapride (4-amino-N-[1-(cyclohex-3-en-1-ylmethyl) piperidin-4-yl]-2-ethoxy-5-nitrobenzamide), metoclopramide (4-amino-5-chloro-N-(2-(diethylamino)ethyl)-2-methoxybenzamide), renzapride (ATL-1251, BRL 24924, (±)-endo-4-amino-5-chloro-2-methoxy-N-(1-azabicyclo[3.3.1]non-4-yl) benzamide), RQ-00000010 (4-{[4-({[4-(2,2,2-trifluoroethoxy)-1,2-benzisoxazol-3-yl]oxy}methyl)piperidin-1-yl]methyl}tetrahydro-2H-pyran-4-carboxylic acid), SUVN-D4010 (1-isopropyl-3-{5-[1-(3-methoxy propyl)piperidin-4-yl]-[1,3,4]oxadiazol-2-yl}-1H-indazole), TD-8954 (4-{(4-[(2-isopropyl-1H-benzoimidazole-4-carbonyl)amino]methyl}-piperidin-1-ylmethyl)piperidine-1-carboxylic acid methyl ester), SC53116 (4-Amino-5-chloro-N-[[(1S,7aS)-hexahydro-1H-pyrrolizin-1-yl]methyl]-2-methoxy-benzamide), BIMU-1 (3-ethyl-2,3-dihydro-N-(8-methyl-8-azabicyclo[3.2.1]oct-3-yl)-2-oxo-1H-benzimidazole-1-carboxamide hydrochloride), donecopride (MR31147, which is: 1-(4-amino-5-chloro-2-methoxyphenyl)-3-[1-(cyclohexylmethyl)-4-piperidinyl]propan-1-one), LS 650155 (Caeserod, which is: 5-(8-amino-7-chloro-2,3-dihydrobenzo[b][1,4]dioxin-5-yl)-3-(1-phenethylpiperidin-4-yl)-1,3,4-oxadiazol-2(3H)-one hydrochloride), PF-00885706, N-[2-[(1R,8S)-4-[[4-(cyclobutylamino)-5-(trifluoromethyl)pyrimidin-2-yl]amino]-11-azatricyclo[6.2.1.02,7]undeca-2(7),3,5-trien-11-yl]-2-oxoethyl]acetamide, and combinations thereof.

RS-67,333 is a high-affinity 5-HT₄R partial agonist (Eglen et al., 1995). This drug is effective in improving behavioral deficits, decreasing the number of amyloid plaques as well as level of amyloid beta (Aβ) species, and decreasing hippocampal astrogliosis and microgliosis in the 5xFAD mouse model of Alzheimer's disease (AD) (Giannoni et al., 2013). RS67333 is an arylketone. Incorporating an n-butyl group on the piperidine has increased the agonist activity with great effectiveness, optimal selectivity, and excellent bioavailability. Its increased hydrophobicity helps pass the blood-brain barrier, allowing for penetration into the brain (Eglen et al. 1995).

Prucalopride is a selective, high affinity 5-HT₄R agonist (Prins et al., 1999). Prucalopride is a derivative of the family of benzofurans which exhibits increased selectivity for 5-HT₄ receptor but no affinity for the hERG (human Ether-a-go-go Related Gene) channels. In 2018, it was approved by the FDA for chronic constipation and is currently being tested for chronic intestinal pseudo-obstruction. Prucalopride has also been tested in two separate clinical trials to investigate its effects on emotional processing in health volunteers after an acute (e.g., single dose) or chronic (e.g., 1 week) administration (Morris et al., 2017; Zanos and Gould, 2018).

PF-04995274 is a potent, partial 5-HT₄R agonist (Grimwood et al., 2011). A clinical trial was conducted to evaluate PF-04995274, alone or in combination with donepezil, on scopolamine-induced deficits in psychomotor and cognitive function in healthy adults. However, this trial was terminated, but not due to safety concerns. Currently, a clinical trial is underway to test whether adjunctive administration of PF-04995247 has positive effects on emotional processing and neural activity in mediated, treatment-resistant (TRD) depressed patients compared to placebo (Morris et al., 2017).

Tegaserod is a partial agonist of the 5-HT₄R, with moderate affinity for the 5-HT₁ (agonist) and 5-HT_{2A-C} (antagonist) receptors. Cisapride is a parasympathomimetic which, by activating the 5-HT₄R, increases the acetylcholine liberated in the enteric nervous system. Cinitapride is a benzamide which acts as a 5-HT_{1A} and 5-HT₄ receptor agonist, and a 5-HT_{2A} receptor antagonist. Mosapride is a selective 5-HT₄R agonist, the main active metabolite of which acts as a 5-HT₃ receptor antagonist. Metoclopramide is a 5-HT₄ and 5-HT_{3A} receptor agonist. It is a D2 receptor antagonist. It is also an M1 muscarinic receptor agonist, and an acetylcholinesterase inhibitor. SUVN-D4010 is a powerful, selective, and effective 5-HT4R partial agonist, having good bioavailability via the oral route.

Mixed 5-HTR agonists/antagonists include, but are not limited to, buspirone, mianserin, trazodone, and mirtazapine.

The terms “serotonin,” “5-hydroxytryptamine” and “5-HT” refers to a phenolic amine neurotransmitter produced from tryptophan by hydroxylation and decarboxylation in serotonergic neurons of the central nervous system and enterochromaffin cells of the gastrointestinal tract. Serotonin is a precursor of melatonin.

### (R,S)-Ketamine

(R,S)-ketamine ((RS)-2-(2-chlorophenyl)-2-(methylamino)cyclohexanone) is an antagonist of the glutamate N-methyl-D-aspartate (NMDA) receptor (NMDAR). (R,S)-ketamine also acts on opioid receptors, sigma receptors, muscarinic receptors, and monoamine transporters.

Ketamine is a chiral compound. As used herein, the term “ketamine” may refer to (S)-ketamine (also referred to as S(+)-ketamine or esketamine), (R)-ketamine (R(−)-ketamine), or a racemic mixture of (S)-ketamine and (R)-ketamine. In certain embodiments, the ketamine compositions contain different proportions of the S(+) and R(−) stereoisomers. In certain embodiments, the ketamine compositions contain only (S)-ketamine or (R)-ketamine or are enantiomerically enriched for a ketamine enantiomer. In certain embodiments, the ketamine composition is enriched to contain, for example, greater than 60%, greater than 70%, greater than 80%, greater than 90%, greater than 95%, greater than 99%, or greater than 99.9 of (S)-ketamine or (R)-ketamine. See Paul et al., 2009; Paskalis et al., 2010; Noppers et al., 2011; Matthews et al., 2012 and PCT Patent Application Publication No. WO 2013/138322.

(R,S)-ketamine is a derivative of arylcyclohexylamine and contains a chiral center. Since the 1950s, a large number of arylcyclohexylamines have been synthesized: these compounds have shown a wide range of possible pharmacological activities. When administered orally, it undergoes first-pass metabolism, where it is stereo selectively metabolized into a broad array of metabolites, including norketamine, hydroxyketamines, dehydronorketamine and hydroxynorketamine (HNK). After (R,S)-ketamine administration, (2S,6S;2R,6R)-HNK are the two major HNK metabolites found in the plasma and brain. Interestingly, a study has shown that the (2R,6R)-HNK metabolite is: 1) essential for the antidepressant effects of ketamine; 2) dependent on α-amino-3-hydroxy-5-methyl-4-isoxazolepropionic acid (AMPA) receptor activation; and 3) non-hypnotic (Zanos et al., 2016). All of these compounds are expected to behave similarly in the presently described methods, including enantiomers and non-psychotomimetic metabolites of ketamine.

The present disclosure also encompasses ketamine's enantiomers and non-psychotomimetic metabolites. Such compounds include:
(2R,6R)-HNK, a metabolite of ketamine that may mediate the antidepressant effects of ketamine and lacks the ketamine-related side effects (Zanos et al., 2016);
(2S,6S)-HNK, a metabolite of ketamine (synthesis of these compounds (2R,6R)-HNK and (25,6S)-HNK are described in Zanos et al., 2016 and PCT Patent Application Publication No. WO 2013/056229. The use of (2R,6R)-hydroxynorketamine, (S)-dehydronorketamine and other stereoisomeric dehydro and hydroxylated metabolites of (R,S)-ketamine in the treatment of depression and neuropathic pain);
(R)-ketamine, the R-enantiomer of ketamine, which has rapid-onset and sustained antidepressant effects without psychotomimetic side effects (Yang et al., 2015); and
(S)-ketamine, the S-enantiomer of ketamine, which is being developed as an intranasal spray, currently in phase III clinical trials for treatment-resistant depression.

Finally, other ketamine analogs are also expected to be protective. Such compounds include: Fluorodeschloroketamine, an analog of ketamine where the chlorine (Cl) group has been replaced by fluorine (F); and Tiletamine, an analog of ketamine commonly used as a veterinary anesthetic.

### NMDA Receptor antagonists-Ketamine and Other Compounds

NMDA receptor antagonists are compounds that antagonize, or inhibit, the action of the NMDA receptor. An NMDA receptor antagonist may be a competitive antagonist, an uncompetitive antagonist, a noncompetitive antagonist, and/or a glycine antagonist.

Non-limiting examples of NMDA receptor antagonists include, ketamine, dextromethorphan (DXM), histogranin, memantine, meperidine, methadone, methoxetamine (MXE), phencyclidine (PCP), nitrous oxide (N₂O), AP5 (APV, R-2-amino-5-phosphonopentanoate), AP7 (2-amino-7-phosphonoheptanoic acid), CPPene ((3-[(R)-2-carboxypiperazin-4-yl]-prop-2-enyl-1-phosphonic acid), Selfotel, Amantadine, Atomoxetine, AZD6765, Agmatine, chloroform, dextrallorphan, dextromethorphan, dextrorphan, diphenidine, dizocilpine (MK-801), ethanol, eticyclidine, gacyclidine, ibogaine, magnesium, memantine, nitromemantine, rolicyclidine, tenocyclidine, methoxydine, tiletamine, neramexane, eliprodil, dexoxadrol, etoxadrol, remacemide, delucemine, WMS-2539, NEFA, 8A-PDHQ, HU-211, Aptiganel (Cerestat, CNS-1102), rhynchophylline, kynurenic acid, Rapastinel (GLYX-13), NRX-1074, 7-Chlorokynurenic acid, 4-Chlorokynurenine (AV-101), TK-40, 1-Aminocyclopropanecarboxylic acid (ACPC), L-Phenylalanine, Xenon, or analogs or derivatives thereof. Ketamine derivatives such as Rapastinel (GLYX-13) are also included. Rapastinel is an NMDA receptor glycine site partial agonist. It is an amidated tetrapeptide (Thr-Pro-Pro-Thr-NH₂) which rapidly crosses the blood brain barrier, but is not active orally.

Compounds that are mechanistically similar to ketamine are expected to be protective against stress-induced de novo psychopathology. Such compounds include:
Ro 25-6981, a GluN2B-selective antagonist, that has been shown to have rapid antidepressant actions in rodent models of depression;
CP-101,606, a GluN2B-selective antagonist: A placebo-controlled trial of the NR2B specific NMDA antagonist CP-101, 606 plus paroxetine for treatment resistant depression (TRD);
GLYX-13, a novel N-methyl-D-aspartate receptor (NMDAR) glycine-site functional partial agonist and rapid-acting antidepressant. GLYX-13 received Breakthrough Therapy designation from the U.S. Food and Drug Administration (FDA) for adjunctive treatment of MDD in January 2016; and
CX546 (Tocris), an ampakine (an AMPA receptor agonist), which relieves the respiratory depression induced by fentanyl.

Non-limiting examples of the NMDA receptor antagonists also include anti-receptor antibodies, and anti-ligand antibodies.

Several synthetic opioids function as NMDA receptor-antagonists, such as pethidine, methadone, meperidine, dextropropoxyphene, tramadol, levorphanol, and ketobemidone.

### AMPA Receptor Agonists

AMPA receptor agonists are compounds that activate the action of the α-amino-3-hydroxy-5-methyl-4-isoxazolepropionic acid (AMPA) receptor. It is expected that compounds that activate the AMPA receptor, including metabolites, will have a similar effect as the present effects shown with ketamine, in view of findings that a ketamine metabolite's antidepressant activity in mice was due to sustained activation of the AMPA receptor, rather than inhibiting NMDAR (Zanos et al., 2016).

Thus, in certain embodiments, AMPA receptor agonists may be used in the methods described herein. Non-limiting examples of the AMPA receptor agonists include glutamate, AMPA, 5-fluorowillardiine, domoic acid, quisqualic acid, (2R,6R)-hydroxynorketamine, and CX546, as well as pharmaceutically acceptable salts, derivatives, or metabolites thereof

### Pharmaceutical Compounds

The 5-HT₄R agonists, (R,S)-ketamine, NMDAR antagonists and AMPAR agonists used in the present methods include all hydrates, solvates, and complexes of the compounds described herein. If a chiral center or another form of an isomeric center is present in a present compound, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present disclosure may be in racemic form or as individual enantiomers. The enantiomers can be separated using known techniques, such as those described in IUPAC (1997) Pure and Applied Chemistry 69:1469-1474. In cases in which compounds have unsaturated carbon-carbon double bonds, both the cis (Z) and trans (E) isomers are within the scope of this disclosure. In cases wherein compounds may exist in tautomeric forms, such as keto-enol tautomers, each tautomeric form is contemplated as being included within this disclosure whether existing in equilibrium or predominantly in one form.

When the structure of the compounds used in this disclosure includes an asymmetric carbon atom such compound can occur as racemates, racemic mixtures, and isolated single enantiomers. All such isomeric forms of these compounds are expressly included in this disclosure. Each stereogenic carbon may be of the R or S configuration. It is to be understood accordingly that the isomers arising from such asymmetry (e.g., all enantiomers and diastereomers) are included within the scope of this disclosure, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The present disclosure is also intended to include use of all isotopes of atoms occurring on the compounds disclosed herein. Isotopes include those atoms having the same atomic number but different mass numbers. Isotopically-labeled compounds can generally be prepared by conventional techniques known to those skilled in the art or by processes analogous to those described herein using appropriate isotopically-labeled reagents in place of the non-labeled reagents employed.

The compounds of the instant disclosure may be in a salt form. As used herein, a “salt” is a salt of the instant compound which has been modified by making acid or base, salts of the compounds. In the case of compounds used for treatment of mammals, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately treating a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The present methods also encompass administering a physiologically functional derivative of the present compound. As used herein, the term “physiologically functional derivative” refers to a compound (e.g., a drug precursor) that is transformed in vivo to yield the present compound or its active metabolite, or a pharmaceutically acceptable salt, hydrate or solvate of the compound. The transformation may occur by various mechanisms (e.g., by metabolic or chemical processes), such as, for example, through hydrolysis in blood. Prodrugs are such derivatives, and a discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

### Pharmaceutical Compositions

While it is possible that the 5-HT₄R agonists, (R,S)-ketamine, a (R,S)-ketamine analog, or a pharmaceutically acceptable salt, derivative, or metabolite thereof, NMDAR antagonists and AMPAR agonists, as well as salts, solvates and physiological functional derivatives thereof, may be administered as the raw chemical, it is possible to present the active ingredient as a pharmaceutical composition. Accordingly, the disclosure further provides a pharmaceutical composition, which comprises the present agent or compound and/or salts, solvates and physiological functional derivatives thereof, and one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In accordance with another aspect of the disclosure there is also provided a process for the preparation of a pharmaceutical composition including admixing the present compound, or salts, solvates and physiological functional derivatives thereof, with one or more pharmaceutically acceptable carriers, diluents or excipients.

The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound or compounds, and pharmaceutically acceptable excipients.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

Pharmaceutical compositions of the present disclosure may be presented in unit dose forms containing a predetermined amount of active ingredient per unit dose. Such a unit may contain, for example, 5 μg to 1 g, preferably 1 mg to 700 mg, more preferably 5 mg to 100 mg of the present compound, depending on the condition being treated, the route of administration and the age, weight and condition of the patient. Such unit doses may therefore be administered more than once a day. Preferred unit dosage compositions are those containing a daily dose or sub-dose (for administration more than once a day), as herein above recited, or an appropriate fraction thereof, of an active ingredient. Furthermore, such pharmaceutical compositions may be prepared by any of the methods well known in the pharmacy art.

Pharmaceutical compositions of the disclosure may be adapted for administration by any appropriate route, for example by the oral (including buccal or sublingual), inhaled, nasal, ocular, or parenteral (including intravenous and intramuscular) route. The present composition may be injected. Such compositions may be prepared by any method known in the art of pharmacy, for example by bringing into association the active ingredient with the carrier(s) or excipient(s).

In a further embodiment, the present disclosure provides a pharmaceutical composition adapted for administration by the oral route.

Pharmaceutical compositions of the present disclosure which are adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free-flowing inert carrier and compressed into tablets directly without going through the granulating or slugging steps. A clear or opaque protective coating consisting of a scaling coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

It should be understood that, in addition to the ingredients particularly mentioned above, the compositions may include other agents conventional in the art having regard to the type of formulation in question, for example those suitable for oral administration may include flavoring agents.

A further preferred form of administration is parenteral including intravenous administration. Pharmaceutical compositions adapted for parenteral administration, including intravenous administration, include aqueous and non-aqueous sterile injectable solutions or suspensions, which may contain anti-oxidants, buffers, bacteriostats, and solutes that render the compositions substantially isotonic with the blood of the subject. Other components which may be present in such compositions include water, alcohols, polyols, glycerin, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

Further methods of administration include mucosal, such as nasal, sublingual, vaginal, buccal, or rectal; or transdermal administration to a subject.

Pharmaceutical compositions adapted for nasal and pulmonary administration may comprise solid carriers such as powders, which can be administered by rapid inhalation through the nose. Compositions for nasal administration may comprise liquid carriers, such as sprays or drops. Alternatively, inhalation directly through into the lungs may be accomplished by inhalation deeply or installation through a mouthpiece. These compositions may comprise aqueous or oil solutions of the active ingredient. Compositions for inhalation may be supplied in specially adapted devices including, but not limited to, pressurized aerosols, nebulizers or insufflators, which can be constructed so as to provide predetermined dosages of the active ingredient.

### Dosages

In certain embodiments, the effective or therapeutically effective amount or dose of the 5-HT₄R agonist, (R,S)-ketamine, a (R,S)-ketamine analog, or a pharmaceutically acceptable salt, derivative, or metabolite thereof, NMDAR antagonist and AMPAR agonist is about 0.01 to about 40 mg per kilogram of body weight of the subject (mg/kg), i.e., from about 0.01 mg/kg to about 40 mg/kg body weight.

In certain embodiments, the effective or therapeutically effective amount or dose of the 5-HT₄R agonist, (R,S)-ketamine, (R,S)-ketamine analog, or a pharmaceutically acceptable salt. derivative, or metabolite thereof. NMDAR antagonist and AMPAR agonist ranges from about 0.01 to about 40 mg/kg body weight, from about 0.01 to about 35 mg/kg body weight, from about 0.01 to about 30 mg/kg body weight, from about 0.01 to about 25 mg/kg body weight, from about 0.01 to about 20 mg/kg body weight, from about 0.01 to about 15 mg/kg body weight, from about 0.01 to about 10 mg/kg body weight, from about 0.01 to about 5 mg/kg body weight, from about 0.01 mg/kg to about 3 mg/kg body weight, from about 0.01 to about 2 mg/kg of body weight, from about 0.01 to about 1.5 mg/kg of body weight, or from about 0.01 to about 1 mg/kg of body weight.

In certain embodiments, the effective or therapeutically effective amount or dose of the 5-HT₄R agonist, (R,S)-ketamine, (R,S)-ketamine analog, or a pharmaceutically acceptable salt, derivative, or metabolite thereof, NMDAR antagonist and AMPAR agonist ranges from about 0.5 to about 40 mg/kg body weight, from about 0.5 to about 35 mg/kg body weight, from about 0.5 to about 30 mg/kg body weight, from about 0.5 to about 25 mg/kg body weight, from about 0.5 to about 20 mg/kg body weight, from about 0.5 to about 15 mg/kg body weight, from about 0.5 to about 10 mg/kg body weight, from about 0.5 to about 5 mg/kg body weight, from about 0.5 mg/kg to about 3 mg/kg body weight, from about 0.5 to about 2 mg/kg of body weight, from about 0.5 to about 1.5 mg/kg of body weight, or from about 0.5 to about 1 mg/kg of body weight.

In certain embodiments, the effective or therapeutically effective amount or dose of the 5-HT₄R agonist, (R,S)-ketamine, (R,S)-ketamine analog, or a pharmaceutically acceptable salt, derivative, or metabolite thereof. NMDAR antagonist and AMPAR agonist ranges from about 1 to about 40 mg/kg body weight, from about 1 to about 35 mg/kg body weight, from about 1 to about 30 mg/kg body weight, from about 1 to about 25 mg/kg body weight, from about 1 to about 20 mg/kg body weight, from about 1 to about 15 mg/kg body weight, from about 1 to about 10 mg/kg body weight, from about 1 to about 5 mg/kg body weight, from about 1 mg/kg to about 3 mg/kg body weight, or from about 1 to about 2 mg/kg of body weight.

In certain embodiments, the effective or therapeutically effective amount or dose of the 5-HT₄R agonist, (R,S)-ketamine, (R,S)-ketamine analog, or a pharmaceutically acceptable salt. derivative, or metabolite thereof, NMDAR antagonist and AMPAR agonist ranges from about 5 to about 40 mg/kg body weight, from about 5 to about 35 mg/kg body weight, from about 5 to about 30 mg/kg body weight, from about 5 to about 25 mg/kg body weight, from about 5 to about 20 mg/kg body weight, from about 5 to about 15 mg/kg body weight, or from about 5 to about 10 mg/kg body weight.

In certain embodiments, the effective or therapeutically effective amount or dose of the 5-HT₄R agonist, (R,S)-ketamine, (R,S)-ketamine analog, or a pharmaceutically acceptable salt, derivative, or metabolite thereof. NMDAR antagonist and AMPAR agonist ranges from about 10 to about 40 mg/kg body weight, from about 10 to about 35 mg/kg body weight, from about 10 to about 30 mg/kg body weight, from about 10 to about 25 mg/kg body weight, from about 10 to about 20 mg/kg body weight, or from about 10 to about 15 mg/kg body weight.

In certain embodiments, the effective or therapeutically effective amount or dose is about 0.01 mg/kg body weight, about 0.05 mg/kg body weight, about 0.1 mg/kg body weight, about 0.2 mg/kg body weight, about 0.3 mg/kg body weight, about 0.4 mg/kg body weight, about 0.5 mg/kg body weight, about 0.6 mg/kg body weight, about 0.7 mg/kg body weight, about 0.8 mg/kg body weight, about 0.9 mg/kg body weight, about 1.0 mg/kg body weight, about 1.1 mg/kg body weight, about 1.2 mg/kg body weight, about 1.3 mg/kg body weight, about 1.4 mg/kg body weight, about 1.5 mg/kg body weight, about 1.6 mg/kg body weight, about 1.7 mg/kg body weight, about 1.8 mg/kg body weight, about 1.9 mg/kg body weight, about 2.0 mg/kg body weight, about 3.0 mg/kg body weight, about 5.0 mg/kg body weight, about 10.0 mg/kg body weight, about 15.0 mg/kg body weight, about 20.0 mg/kg body weight, about 25.0 mg/kg body weight, about 30.0 mg/kg body weight, about 35.0 mg/kg body weight, about 40.0 mg/kg body weight, or a number or a range between any of these values.

In certain embodiments, the effective or therapeutically effective amounts or doses listed above are the amounts of the individual 5-HT₄R agonist, (R,S)-ketamine, (R,S)-ketamine analog, or a pharmaceutically acceptable salt, derivative, or metabolite thereof, NMDAR antagonist and AMPAR agonist. In some embodiments, the effective or therapeutically effective amounts or doses listed above are the total amounts of the total 5-HT₄R agonist and (R,S)-ketamine, (R,S)-ketamine analog, or a pharmaceutically acceptable salt, derivative, or metabolite thereof. NMDAR antagonist or AMPAR agonist in combination. For example, an effective or therapeutically effective amount or dose can comprise about 0.01 to about 40 mg per kilogram of a 5-HT₄R agonist and about 0.01 to about 40 mg per kilogram of (R,S)-ketamine. In a further example, an effective or therapeutically effective amount or dose can comprise about 0.01 to about 40 mg per kilogram total of a 5-HT₄R agonist combined with (R,S)-ketamine.

In certain embodiments, the dose of the present composition or compositions per administration is from about 1 to about 250 mg, about 10 mg to about 300 mg, about 10 mg to about 250 mg, about 10 to about 200 mg, about 15 to about 175 mg, about 20 to about 175 mg, about 8 mg to about 32 mg, about 50 mg to about 75 mg, about 25 to about 150 mg, about 25 to about 125 mg, about 25 to about 100 mg, about 50 to about 100 mg, about 50 mg to about 75 mg, about 75 mg to about 100 mg, or about 75 mg to about 200 mg, or about 100 mg to about 300 mg, or about 100 mg to about 400 mg, or about 250 mg to about 500 mg.

In certain embodiments, the dose of the present composition or compositions per administration is about 1 mg, 2 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 300 mg, 350 mg, 400 mg, 450 mg, 500 mg, or a number or a range between any of these values.

In certain embodiments, a single composition contains or comprises both active agents or compounds, e.g., 5-HT₄R agonist and ketamine, ketamine analog. or a pharmaceutically acceptable salt, derivative, or metabolite thereof. NMDAR antagonist and/or AMPAR agonist are in a single composition. In some embodiments, the active agents or compounds are in different compositions. In certain embodiments, an effective amount of the ketamine is a sub-anesthetic amount of ketamine, or a pharmaceutically acceptable salt or solvate thereof, or a physiologically functional derivative thereof.

In certain embodiments, the effective or therapeutically effective amount or dose is below the level that results in one or more side effects of the agent.

In certain embodiments, the effective or therapeutically effective amount or dose may be adjusted depending on conditions of the disease/disorder to be treated or prophetically treated, the age, body weight, general health conditions, sex, and diet of the subject, dose intervals, administration routes, excretion rate, and combinations of drugs.

An initial dose may be larger, followed by one or more smaller maintenance doses. Other ranges are possible, depending on the subject's response to the treatment. An initial dose may be the same as, or lower or higher than subsequently administered doses.

The agents, compounds, composition, or compositions may be administered daily, weekly, biweekly, several times daily, semi-weekly, every other day, bi-weekly, quarterly, several times per week, semi-weekly, monthly, or more. The duration and frequency of treatment may depend upon the subject's response to treatment.

In certain embodiments, a subject may be administered 1 dose, 2 doses, 3 doses, 4 doses, 5 doses, 6 doses or more of the present agents, compounds, composition or compositions. In certain embodiments, a single dose of the present agents, compounds, composition or compositions is administered in the present method. In certain embodiments, multiple doses of the present agents, compounds, composition, or compositions (e.g., 2 doses, 3 doses, 4 doses, 5 doses, 6 doses, 7 doses, 8 doses, 9 doses, 10 doses or more) are administered in the present method.

In certain embodiments, when there are more than one doses of the present agents, compounds, composition, or compositions administered to a subject, the second dose is lower than the first dose. In certain embodiments, the second dose is an amount that is at most one-half, one-quarter, or one-tenth the amount of the first dose.

The number and frequency of doses may be determined based on the subject's response to administration of the agents, compounds, composition or compositions, e.g., if one or more of the patient's symptoms improve and/or if the subject tolerates administration of the composition without adverse reaction.

In certain embodiments, the agents, compounds present composition or compositions is administered at least once a day, at least twice a day, at least three times per day, or more. In certain embodiments, the agents, compounds, composition, or compositions is administered at least once a week, at least twice a week, at least three times per week, or more frequently. In certain embodiments, the present composition or compositions is administered at least twice per month, or at least once per month.

Treatment using the present method can continue as long as needed. Some embodiments provided herein contemplate administration of a single agent or compound disclosed herein. For example, in some embodiments of the methods provided herein, either (i) prucalopride or a pharmaceutically acceptable salt, stereoisomer, tautomer, analog, derivative, or metabolite thereof, or (ii) ketamine, a ketamine analog, or a pharmaceutically acceptable salt, stereoisomer, tautomer, derivative, or metabolite thereof, is administered to a subject in need, and said administration can treat Alzheimer's disease or another neurodegenerative disease and/or improve or ameliorate symptoms associated with Alzheimer's disease.

### Combination Therapy

The present agents, compounds, and compositions may be administered to a subject alone or may be administered to a subject in combination with one or more additional agents.

In certain embodiments, the additional agent is a therapeutic or prophylactic agent for Alzheimer's disease or other neurodegenerative diseases, or an agent for ameliorating or treating the symptoms of Alzheimer's disease or other neurodegenerative diseases.

In certain embodiments, combination therapy means simultaneous administration of the agents in the same dosage form, simultaneous administration in separate dosage forms, or separate administration of the agents. For example, in some embodiments of the methods provided herein, (i) prucalopride or a pharmaceutically acceptable salt, stereoisomer, tautomer, analog, derivative, or metabolite thereof, and (ii) ketamine, a ketamine analog, or a pharmaceutically acceptable salt, stereoisomer, tautomer, derivative, or metabolite thereof, are administered: (a) in a single dosage form and/or concurrently; or (b) in separate dosage forms and/or separately.

In certain embodiments, the additional agent is used as adjunctive therapy to the present agents, compounds, composition, or compositions. In certain embodiments, the treatment includes a phase wherein treatment with the additional agent takes place after treatment with the present agents, compounds, composition, or compositions has ceased. In certain embodiments, the treatment includes a phase where treatment with the present agents, compound, composition or compositions and treatment with the additional agent/treatment overlap.

Combination therapy can be sequential or can be administered simultaneously. In either case, these agents, compounds, and compositions are said to be “co-administered.” It is to be understood that “co-administered” does not necessarily mean that the agents, compounds and compositions are administered in a combined form (i.e., they may be administered separately (e.g., as separate compositions or formulations) or together (e.g., in the same formulation or composition) to the same or different sites at the same or different times).

In certain embodiments, a subject is treated concurrently (or concomitantly) with the present agents, compounds, composition or compositions and an additional agent. In certain embodiments, a subject is treated initially with the present agents, compounds, composition or compositions, followed by cessation of the present agents, compounds, composition or compositions and initiation of treatment with an additional agent. In certain embodiments, the present agents, compounds, composition, or compositions are used as an initial treatment, e.g., by administration of one, two or three doses, and an additional agent is administered to prolong the effect of the present agents, compounds, composition, or compositions, or alternatively, to boost the effect of the present agents, compounds, composition or compositions. A person of ordinary skill in the art will recognize that other variations of the presented schemes are possible, e.g., initiating treatment of a subject with the present agents, compounds, composition, or compositions, followed by a period wherein the subject is treated with an additional agent as adjunct therapy to the present agents, compounds, composition or compositions treatment, followed by cessation of the present agents, compounds, composition or compositions treatment.

The present agents, compounds, composition or compositions and the additional pharmaceutically active agent(s) may be administered together or separately and, when administered separately this may occur simultaneously or sequentially in any order. The amounts of the present agents, compounds, composition or compositions and the additional pharmaceutically active agent(s) and the relative timings of administration will be selected in order to achieve the desired combined therapeutic effect.

In various embodiments, the therapies (e.g., agents, compounds, composition or compositions provided herein and an additional agent in a combination therapy) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In certain embodiments, the therapies are administered no more than 24 hours apart or no more than 48 hours apart. In certain embodiments, two or more therapies are administered within the same patient visit. In other embodiments, the agents, compounds, composition, or compositions provided herein and the additional agent are administered concurrently. In other embodiments, the agents, compounds, composition, or compositions provided herein and the additional agent are administered at about 2 to 4 days apart, at about 4 to 6 days apart, at about 1 week part, at about 1 to 2 weeks apart, or more than 2 weeks apart. In certain embodiments, administration of the same agent may be repeated and the administrations may be separated by at least 1 day, 2 days, 3 days, 5 days, 10 days, 15 days, 30 days, 45 days, 2 months, 75 days, 3 months, 6 months. or a number or a range between any of these values. In other embodiments, administration of the same agent may be repeated and the administration may be separated by at least 1 day, 2 days, 3 days, 5 days, 10 days, 15 days, 30 days, 45 days, 2 months, 75 days, 3 months, 6 months, or a number or a range between any of these values.

In certain embodiments, the agents, compounds, composition, or compositions provided herein and a second agent are administered to a subject in a sequence and within a time interval such that the agents, compounds, composition or compositions provided herein can act together with the additional agent to provide an increased benefit than if they were administered otherwise. In one embodiment, the agents, compounds, composition, or compositions provided herein and the additional agent exerts their effect at times which overlap. Each additional agent can be administered separately, in any appropriate form and by any suitable route. In other embodiments, the agents, compounds, composition, or compositions provided herein is administered before, concurrently or after administration of the second active agent. The additional agent can act additively or synergistically with the agents, compounds, composition, or compositions provided herein.

In one embodiment, the agents, compounds, composition, or compositions provided herein is administered concurrently with one or more second agents in the same pharmaceutical composition. In another embodiment, agents, compounds, composition, or compositions composition provided herein is administered concurrently with one or more additional agents in separate pharmaceutical compositions.

Also contemplated are administration of agents, compounds, composition, or compositions provided herein and an additional agent by the same or different routes of administration, e.g., oral and parenteral.

### Kits

Also provided are kits for use in the present methods. The kits can include agents, compounds, composition, or compositions provided herein, and instructions providing information to a health care provider regarding usage in accordance with the present methods. The kit may optionally contain an additional agent or composition. Instructions may be provided in printed form or in the form of an electronic medium such as a floppy disc, CD, or DVD, or in the form of a website address where such instructions may be obtained. A unit dose of agents, compounds, composition, or compositions provided herein can include a dosage such that when administered to a subject, a therapeutically or prophylactically effective plasma level of the compound or composition can be maintained in the subject for at least 1 days. In some embodiments, a compound or composition can be included as a sterile aqueous pharmaceutical composition or dry powder (e.g., lyophilized) composition. In some embodiments, suitable packaging is provided. As used herein, “packaging” includes a solid matrix or material customarily used in a system and capable of holding within fixed limits a compound provided herein and/or a second agent suitable for administration to a subject. Such materials include glass and plastic (e.g., polyethylene, polypropylene, and polycarbonate) bottles, vials, paper, plastic, and plastic-foil laminated envelopes and the like.

The kits described herein contain one or more containers, which contain compounds, signaling entities, biomolecules and/or particles as described. The kits also contain instructions for mixing, diluting, and/or administrating the compounds. The kits also include other containers with one or more solvents, surfactants, preservative and/or diluents (e.g., saline (0.9% NaCl), or 5% dextrose) as well as containers for mixing, diluting, or administering the components to the sample or to the patient in need of such treatment.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are used, the liquid form may be concentrated or ready to use. The solvent will depend on the compound and the mode of use or administration. Suitable solvents for drug compositions are well known and are available in the literature. The solvent will depend on the compound and the mode of use or administration.

The kits comprise a carrier being compartmentalized to receive in close confinement one or more container such as vials, tubes, and the like, each of the container comprising one of the separate elements to be used in the method. For example, one of the container may comprise a positive control in an assay. Additionally, the kit may include containers for other components, for example, buffers useful in the assay.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

### Materials and Methods for Examples 2-5

### Mice

The APP/PS1 (AD) mice were previously bred with the ArcCreER^{T2}×enhanced yellow fluorescent protein (EYFP) mice (Denny et al., 2014; Perusini et al., 2017). Female and male mice were used in all experiments. The Ctrl mice were APP/PS1 (−)×ArcCreER^{T2} (+) or (−)×EYFP (+/−), and the AD mice were APP/PS1 (+)×ArcCreER^{T2} (+) or (−)×EYFP (+/−). Mice were housed 4-5 per cage in a 12-h (06:00-18:00) light-dark colony room at 22° C. Mice had ad libitum access to food and water. All experiments were approved by the Institutional Animal Care and Use Committee (IACUC) at the New York Psychiatric Institute (NYSPI).

### Drugs

All drugs were resuspended in saline and made fresh for each experiment. A single injection of saline (0.9% NaCl), or (R,S)-ketamine (Fort Dodge Animal Health, Fort Dodge, IA) (10 mg/kg or 30 mg/kg), and/or prucalopride (SML1371, Sigma-Aldrich, St. Lois, MO) (1.5 mg/kg or 3 mg/kg), was administered once during the course of each experiment. These dosages were chosen based upon previous studies (Brachman et al., 2016; Chen et al., 2020a). A combination of (R,S,)-ketamine (K)+prucalopride (P) (10 mg/kg of K and 3 mg/kg of P) was administered in a single dose. All drugs were prepared in physiological saline and administered intraperitoneally (i.p.) in volumes of 0.1 cc per 10 mg body weight.

### Behavioral Assays

### Piezo Sleep Boxes

Mice were placed into Piezo sleep boxes (Signal Solutions LLC, Lexington, KY) individually with soft bedding, food, and water for 5 days on a 12 h light/dark cycle. The floor sensors tracked sleep bouts, respiration, temperature, and movements. Sleep/wake recordings were analyzed using the Sleep Statistics Toolbox (Signal Solutions LLC, Lexington, KY), WakeActive and ActivityStatistics code (Ryan Logan, Boston University), and ClockLab (Actimetrics).

### Novelty Suppressed Feeding (NSF)

Testing was performed as previously described (Brachman et al., 2016; Chen et al., 2020a). Briefly, the testing apparatus consisted of a plastic box (50×50×20 cm). The floor was covered with approximately 2 cm of wooden bedding and the arena was brightly lit (approximately 1000 lux). Mice were food restricted for 12 hours prior to testing. At the time of testing, a single pellet of food (regular chow) was placed on a white paper platform positioned in the center of the box. Each animal was placed in a corner of the box, and a stopwatch was immediately started. The latency of the mice to begin eating was recorded. Immediately after the latency was recorded, the food pellet was removed from the arena. The mice were then placed into their home cage and the amount of food consumed in 5 min was measured (home cage consumption), followed by an assessment of post-restriction weight. Kaplan-Meier survival analysis was used due to the lack of normal distribution of data. The Mantel-Cox log-rank test was used to evaluate differences between the experimental groups.

### Marble Burying (MB)

The MB assay was conducted in a clean cage (10.5 in×5.5 in) containing soft pliable Beta Chip bedding (Northeastern Products Corp, Warrensburg, NY). The cage contained 16 marbles set up in 4 rows of 4 across. Mice were given 20 minutes to explore and bury. At the end of the assay, the percentage of marbles buried was calculated.

### Contextual Fear Conditioning (CFC)

A 3-shock CFC paradigm was administered as previously described (Denny et al., 2014; Drew et al., 2010). Fear conditioning was conducted in chambers obtained from Coulborn Instruments (Allentown, PA) with internal dimensions of approximately 20 cm wide×16 cm deep×20.5 cm high. The chambers had metal walls on each side, clear plastic front and back walls and ceilings, and stainless-steel bars on the floor. A house light (CM1820 bulb, 28 V, 100 mA) mounted directly above the chamber provided illumination. Each chamber was located inside a larger, insulated, plastic cabinet that provided protection from outside light and noise. Each cabinet contained a ventilation fan that was operated during the sessions. A paper towel dabbed with lemon solution was placed underneath the chamber floor. Mice were held outside the experimental room in their home cages prior to testing and transported to the conditioning apparatus individually in standard mouse cages. Chambers were cleaned with 70% EtOH between each set of mice. Training sessions were conducted using a 3-shock protocol. Mice were placed into the conditioning chamber and received shocks at 180 s, 240 s, and 300 s (2 s duration each, 0.75 mA). Fifteen seconds after the last shock, mice were removed from the chamber. Overall, the training session lasted 317 s. During re-exposure, mice were placed in the conditioning chamber for 3 minutes and did not receive any shocks. All sessions were scored for freezing using FreezeFrame4 (Actimetrics, Wilmette, IL).

### Statistical Analysis

Data were analyzed using JMP© software (SAS Institute, Cary, NC). Alpha was set to 0.05 for all analyses. Generally, the effect of Drug or Genotype was analyzed using an analysis of variance (ANOVA), using repeated measures where appropriate. Post-hoc Dunnett's or Tukey's tests were used where appropriate. Graphs were made using Prism v8.0 (Graphpad Software, La Jolla, CA).

### Example 2

### Combined (R,S)-Ketamine and Prucalopride Decreased Sleep Amplitude in Male APP/PS1 Mice

It was first sought to determine if combined (R,S)-ketamine and prucalopride would alter sleep behavior in Ctrl and AD male mice (FIG. 1A). Following drug administration, mice were placed in Piezo sleep boxes for 4 days. Average activity was measured across 24 hours throughout the light (0-12 ZT) and dark periods (12-23 ZT). As expected, mice exhibited increased activity during the night. However, activity was decreased significantly in the combined (R,S)-ketamine and prucalopride-treated Ctrl and AD mice (FIGS. 1B-1C).

Amplitude, a measure of differences between sleeping and awake states, was decreased in (R,S)-ketamine and prucalopride-treated AD mice compared to (R,S)-ketamine and prucalopride-treated Ctrl mice (FIG. 1D). There was an overall Time×Genotype×Drug interaction across 4 days. Post-hoc tests did not reveal any significant differences (FIG. 1E). Average sleep percentage was similar among the groups (FIG. 1F).

During the light phase, AD mice exhibited less overall sleep compared to Ctrl mice (FIG. 1G), however, drug administration did not impact this phenotype. Finally, there was no effect of Drug or Genotype during the dark phase (FIG. 1H).

These data suggested that combined (R,S)-ketamine and prucalopride is effective for improving sleep abnormalities in AD.

### Example 3

### Combined (R,S)-Ketamine and Prucalopride Did Not Impact Hyponeophagia

Mice were next tested in a battery of behavioral paradigms to measure hyponeophagia, anxiety and perseverative behavior, and memory. See FIG. 1A.

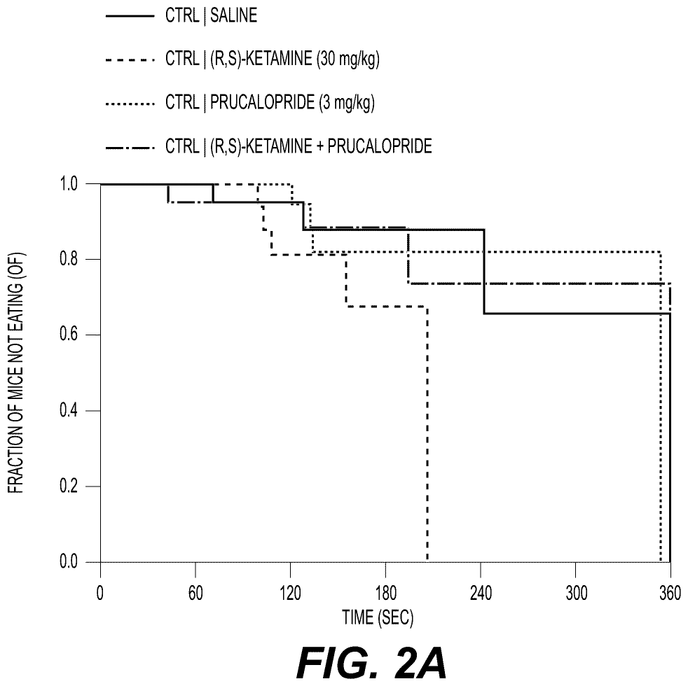

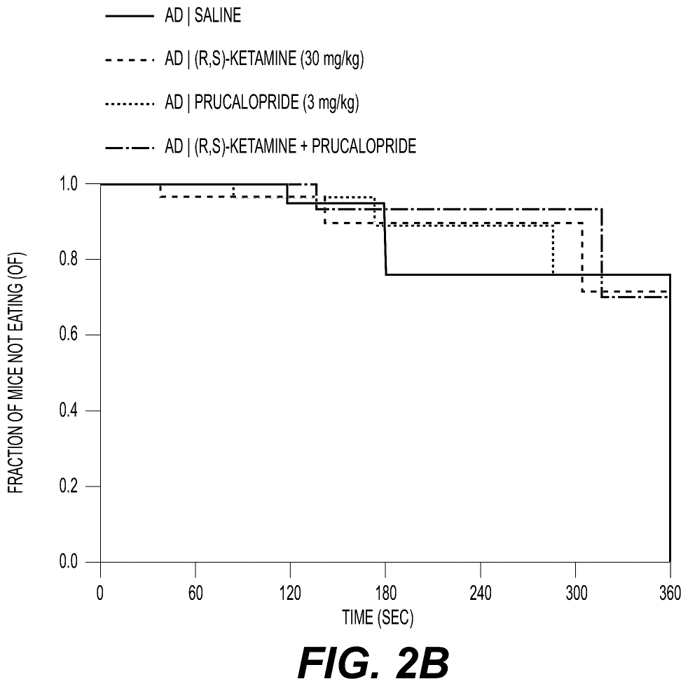

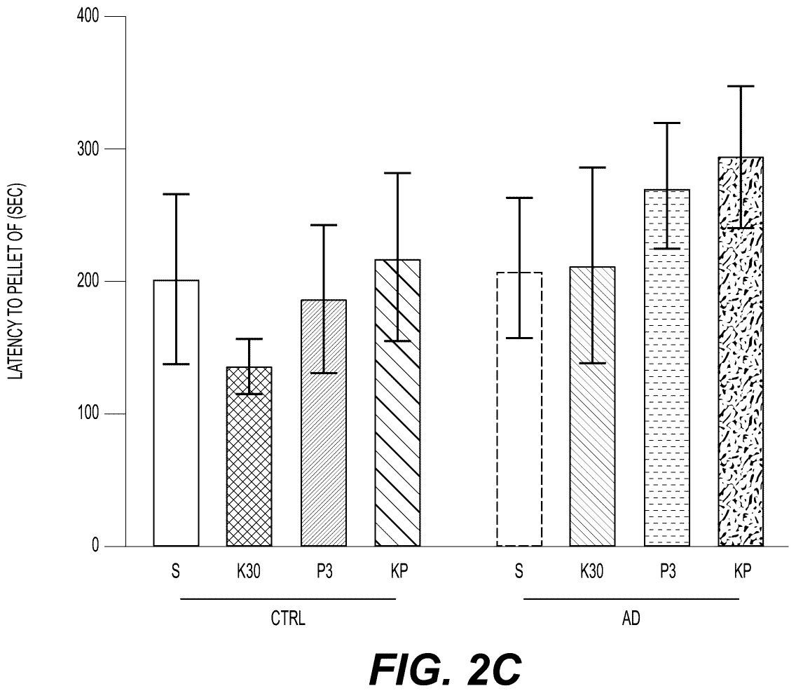

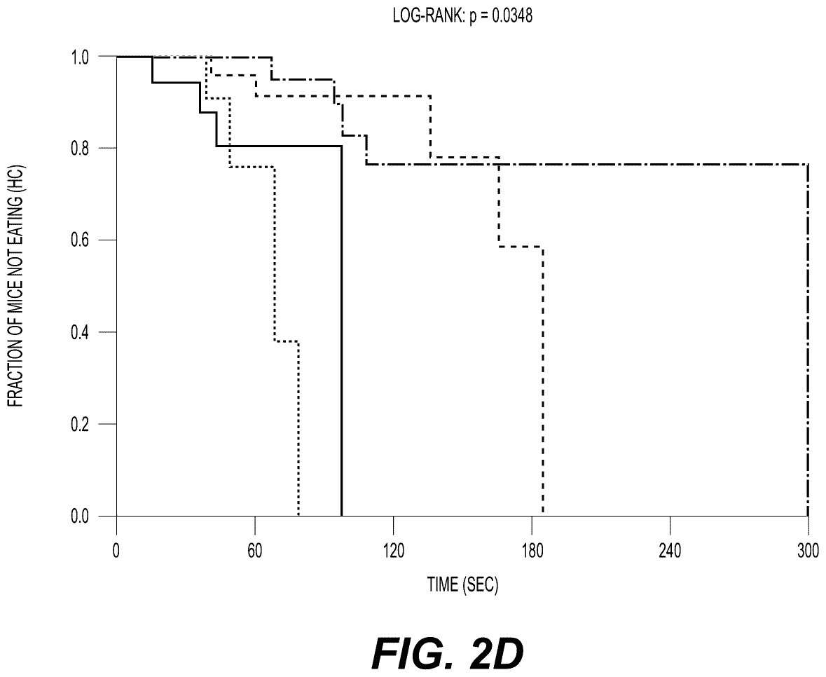

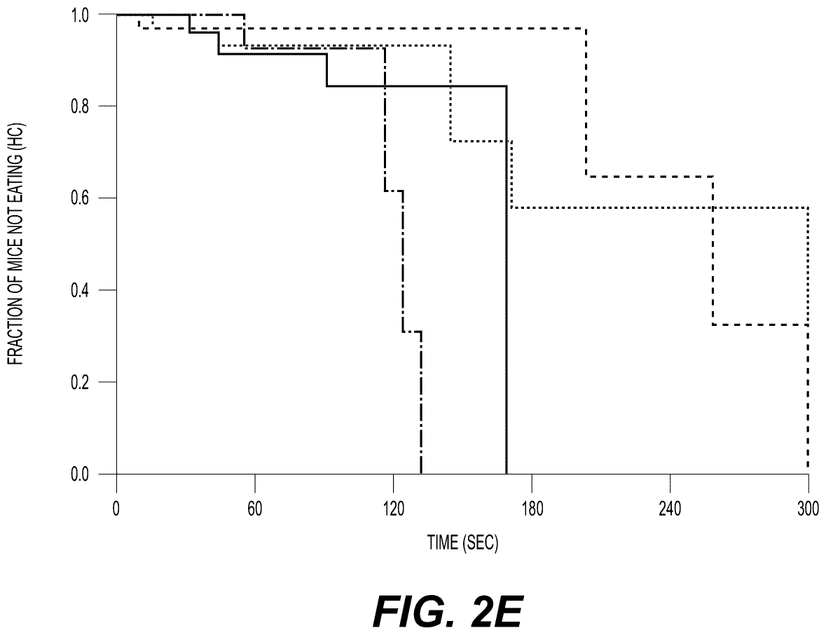

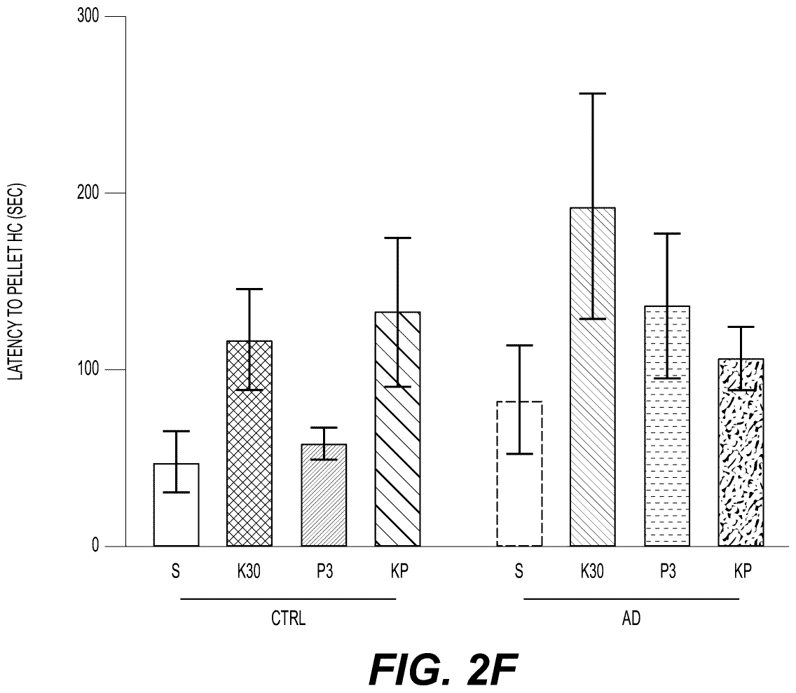

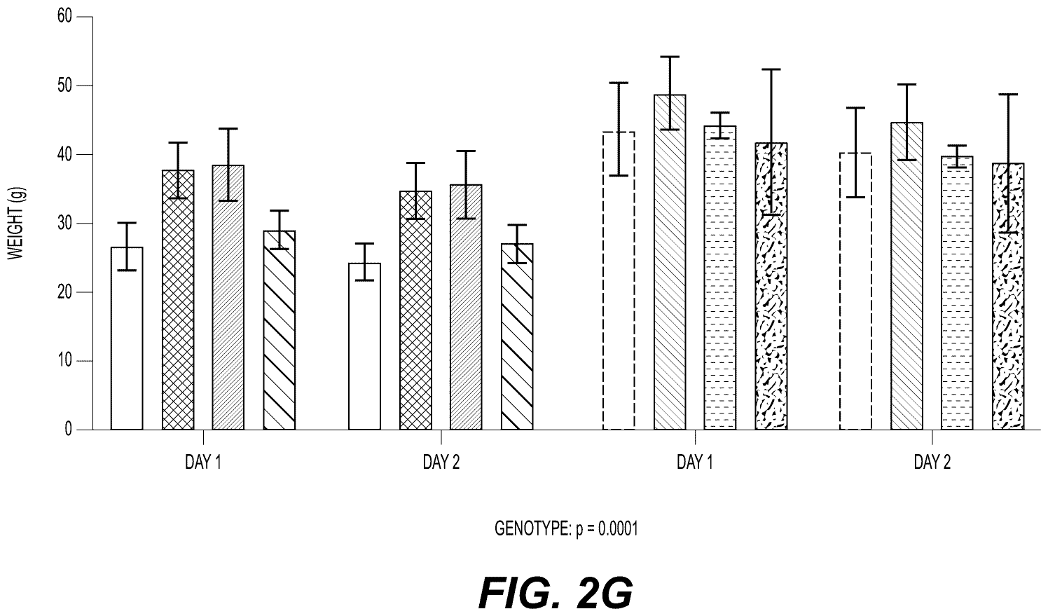

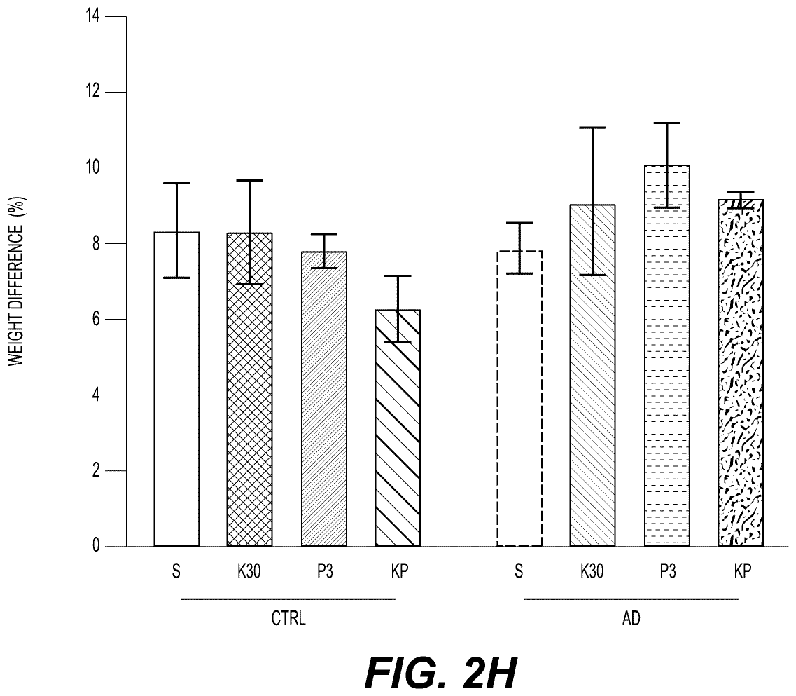

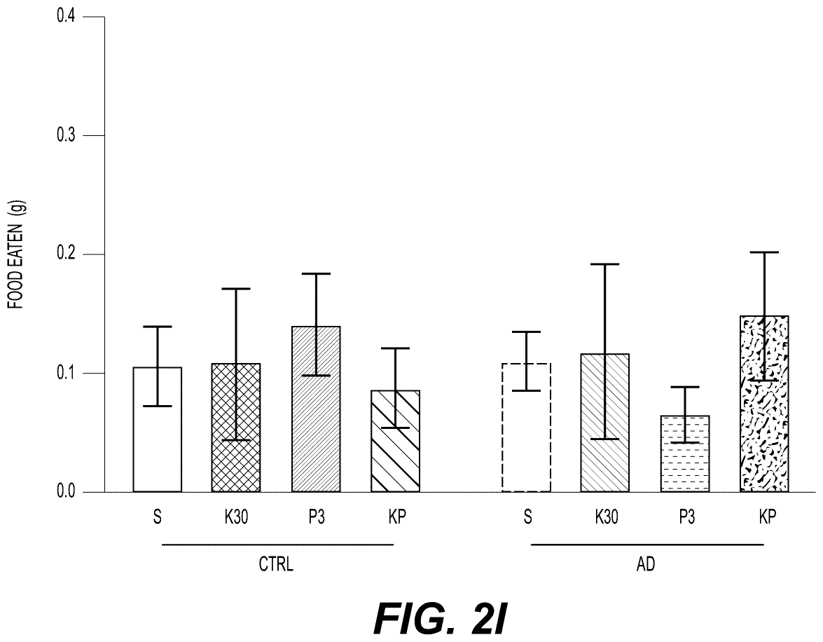

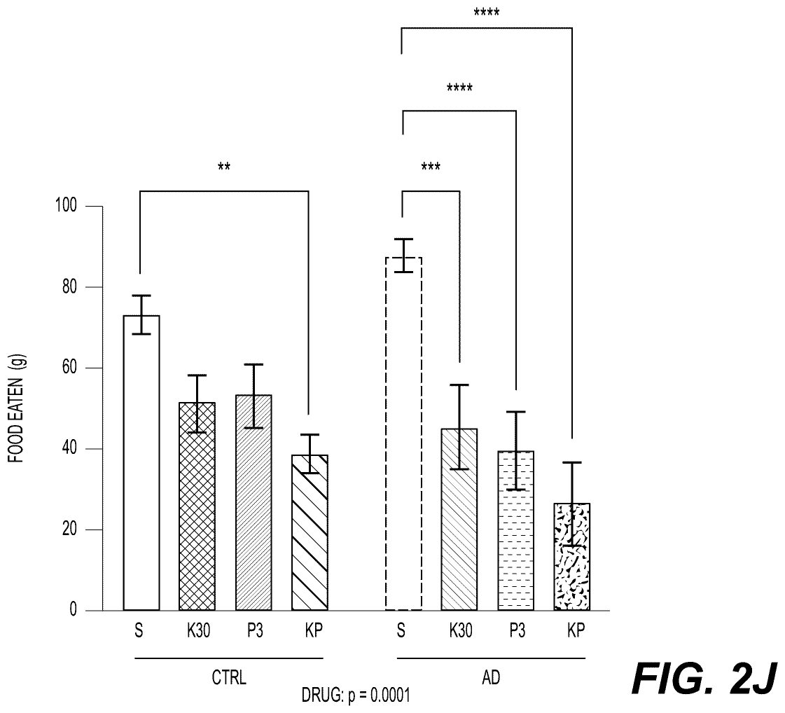

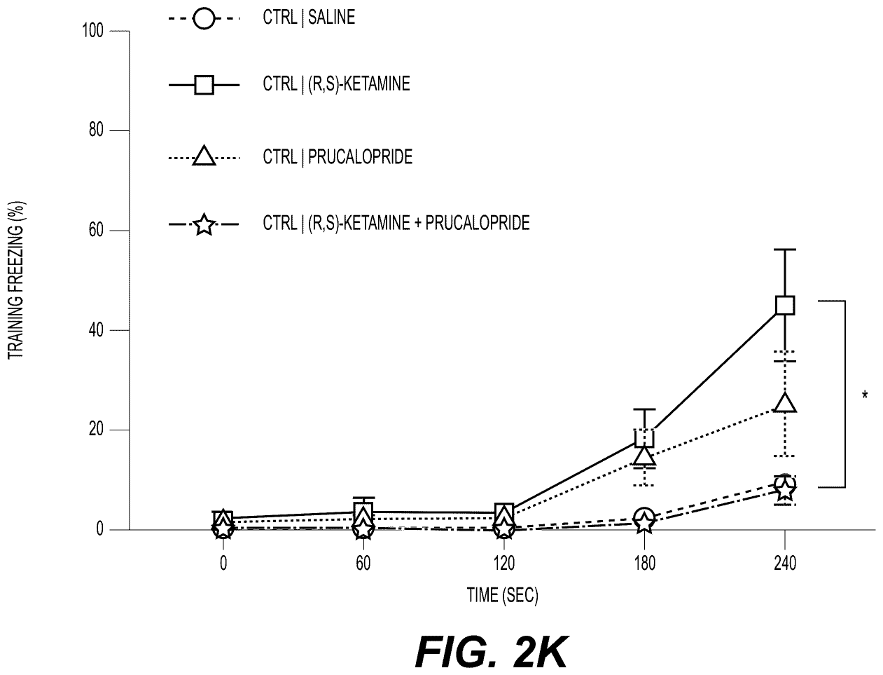

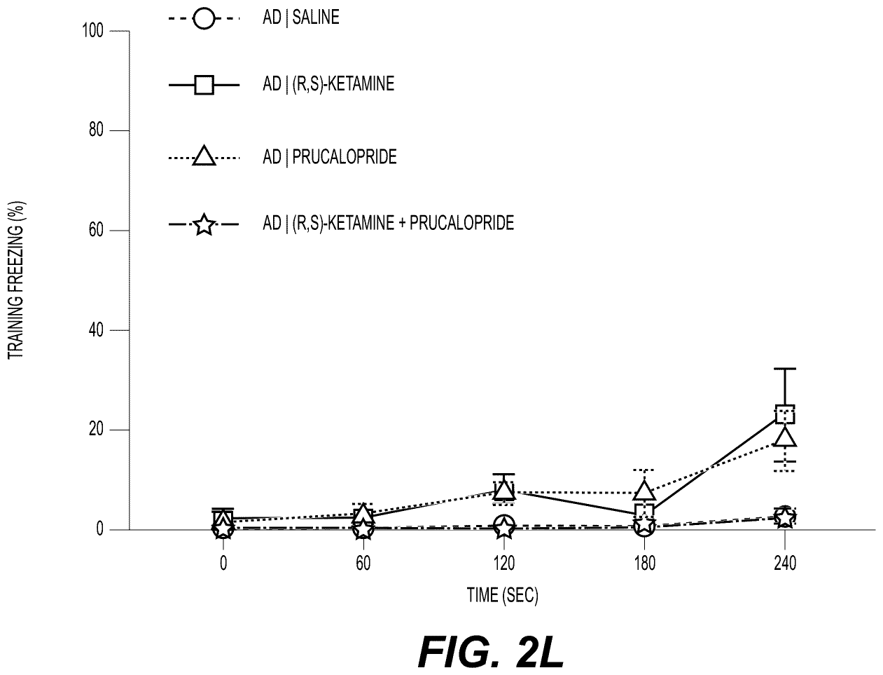

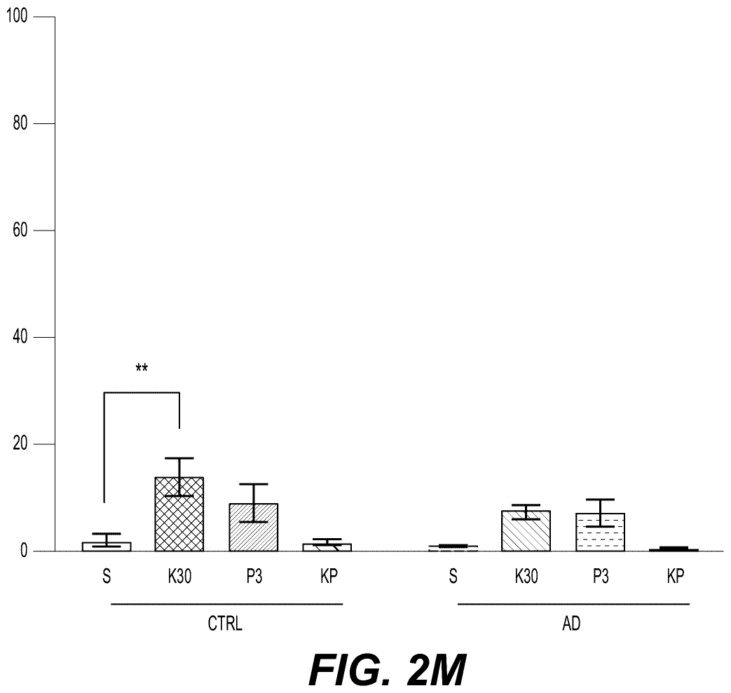

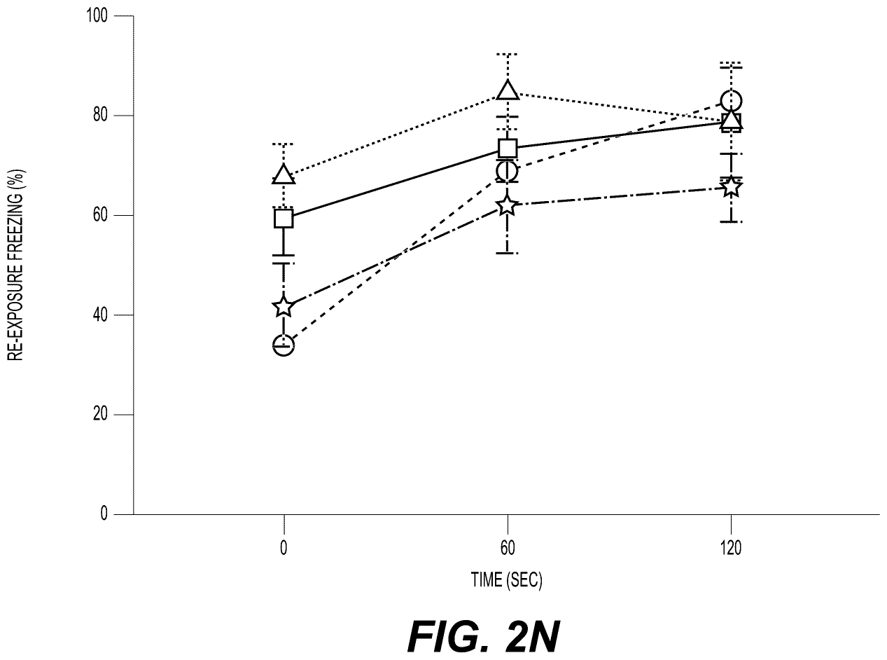

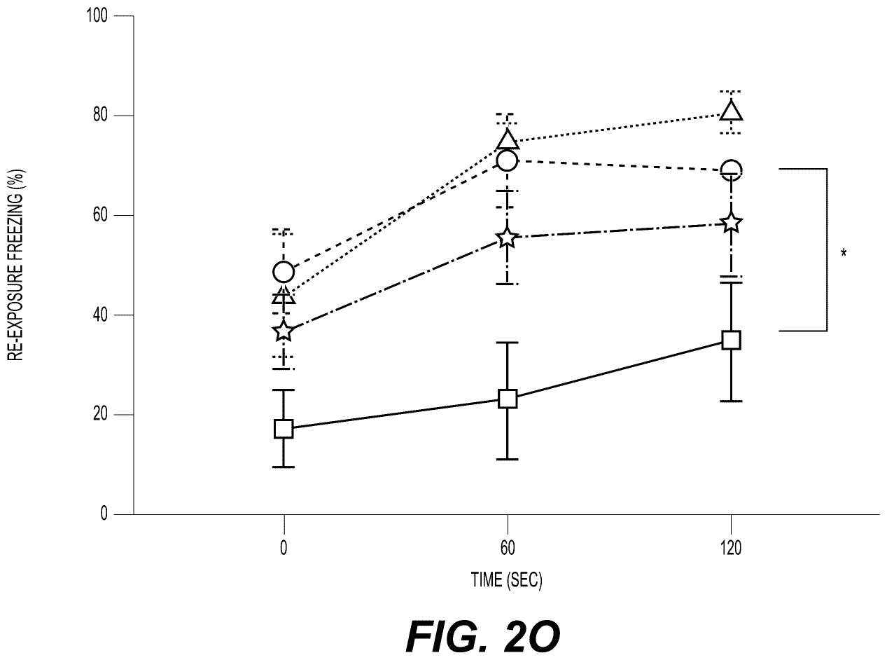

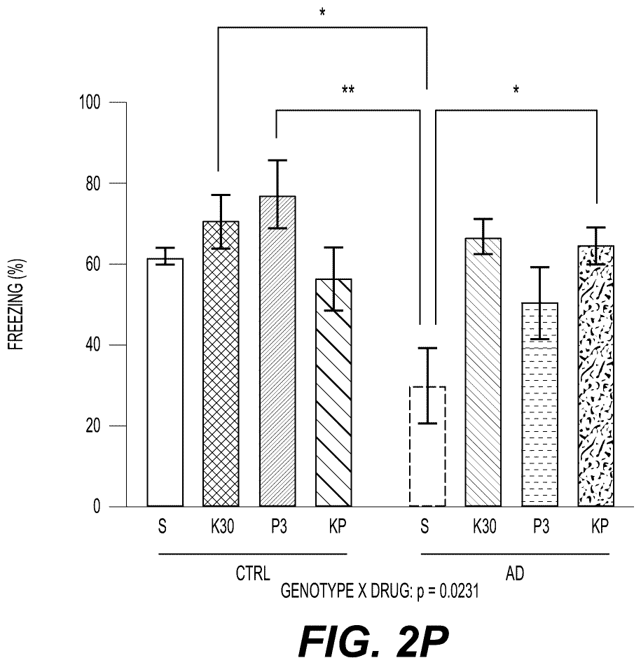

In the NSF paradigm, there were no differences in the latency to approach the food pellet between the groups (FIGS. 2A-2C). However, Ctrl mice treated with prucalopride approached the pellet more quickly when placed in their home cage when compared to Ctrl mice (FIGS. 2D-2F). AD mice weighed more than Ctrl mice (FIG. 2G), but weight nor weight loss was not impacted by Drug (FIGS. 2H-2I). Food consumed in the home cage was similar among the groups (FIG. 2I).

These data suggested that combined (R,S)-ketamine and prucalopride may not be effective for hyponeophagia.

### Example 4

### Combined (R,S)-Ketamine and Prucalopride Decreased Perseverative Behavior

In the MB task, combined (R,S)-ketamine and prucalopride administration, but not single drug administration, decreased perseverative behavior in Ctrl mice (FIG. 2J). However, in AD mice, all drugs reduced perseverative behavior.

These data suggest that combined (R,S)-ketamine and prucalopride is effective for improving perseverative or OCD-like behavior.

### Example 5

### Combined (R,S)-Ketamine and Prucalopride Improved Memory Loss in Male APP/PS1 Mice

Mice were administered a 3-shock CFC paradigm to assay learning and memory. (R,S)-ketamine-treated Ctrl mice treated exhibited increased freezing during training when compared to other groups (FIG. 2K). Drug administration did not impact learning in the AD mice (FIG. 2L-2M). During memory retrieval, all groups of Ctrl mice froze comparably (FIG. 2N). Saline-injected AD mice were impaired in memory retrieval (i.e., decreased freezing) when compared to Ctrl mice (FIG. 2O-2P).

Notably, only combined (R,S)-ketamine and prucalopride administration increased freezing behavior, a proxy for memory retrieval, in AD mice.

These data suggest that combined (R,S)-ketamine and prucalopride is effective for improving memory loss.

### Example 6

Combined prophylactic administration of (R,S)-ketamine and prucalopride administration decreases perseverative behavior in female APP/PS1 mice.

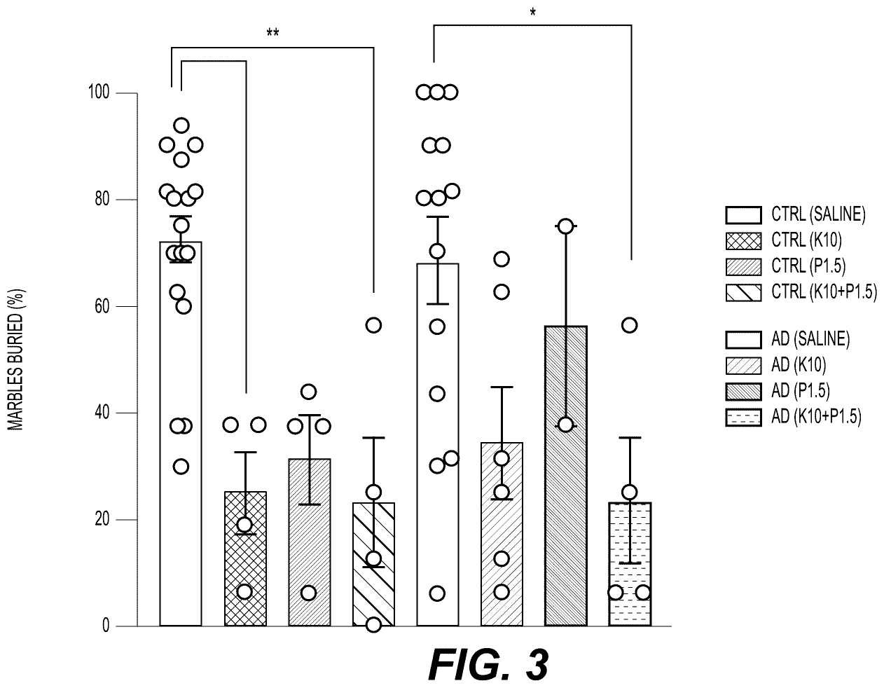

As shown by the number of marbles buried in the MB task, (R,S)-ketamine and combined (R,S)-ketamine and prucalopride administration decreased perseverative behavior in Ctrl mice. In AD mice, combined (R,S)-ketamine and prucalopride decreased perseverative behavior (FIG. 3).

These data suggest that combined (R,S)-ketamine and prucalopride administration is effective for improving perseverative or OCD-like behavior, irrespective of the sex of the subject.

### Example 7

Chronic combined (R,S)-ketamine and prucalopride administration improves memory retrieval in 2-month-old male APP/PS1 mice.

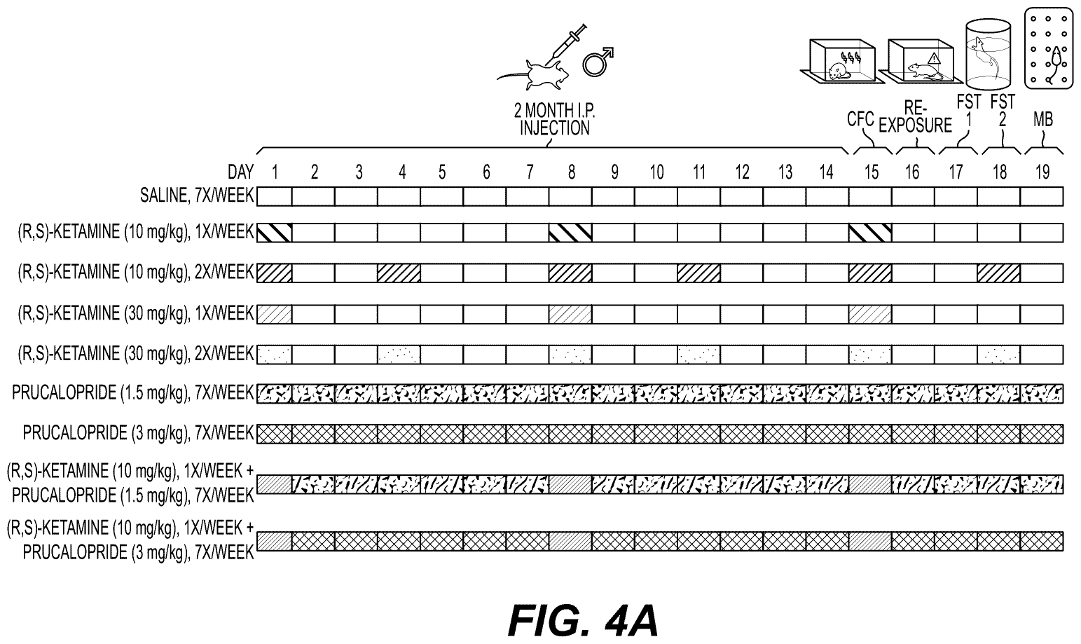

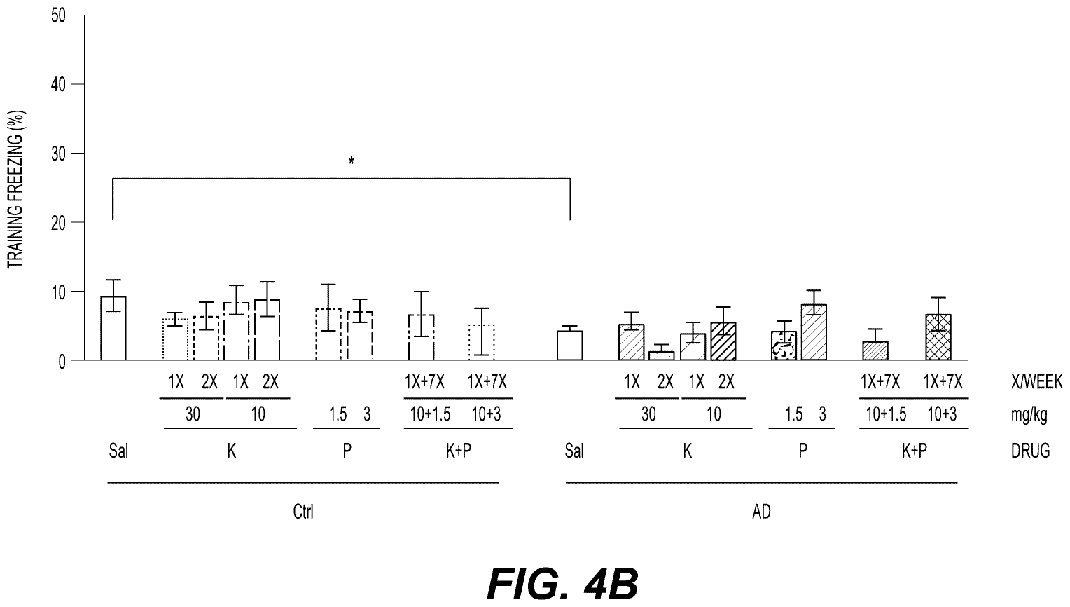

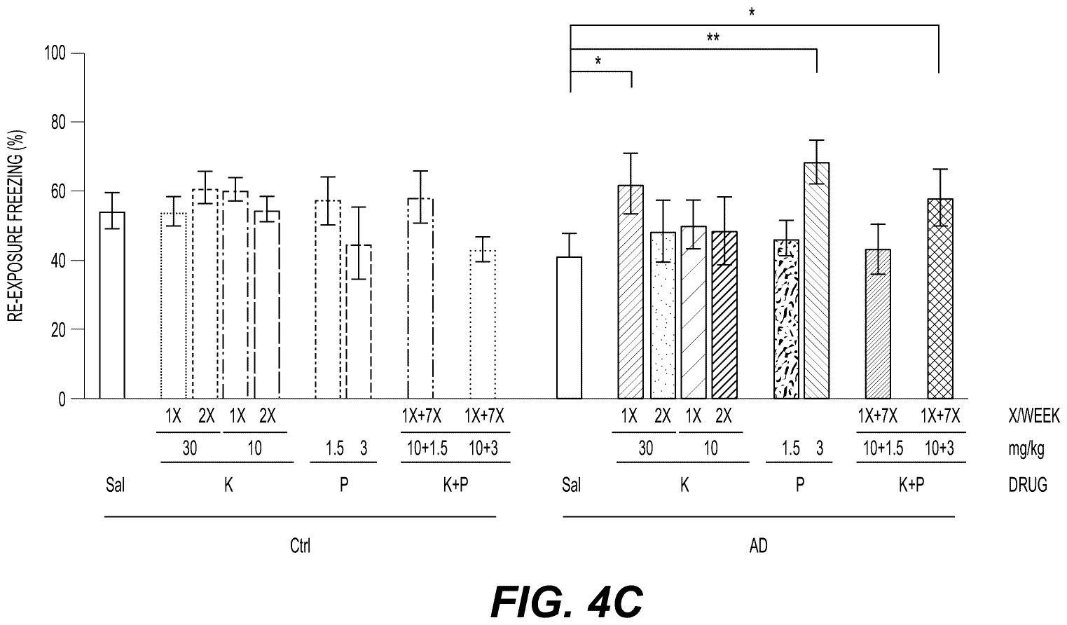

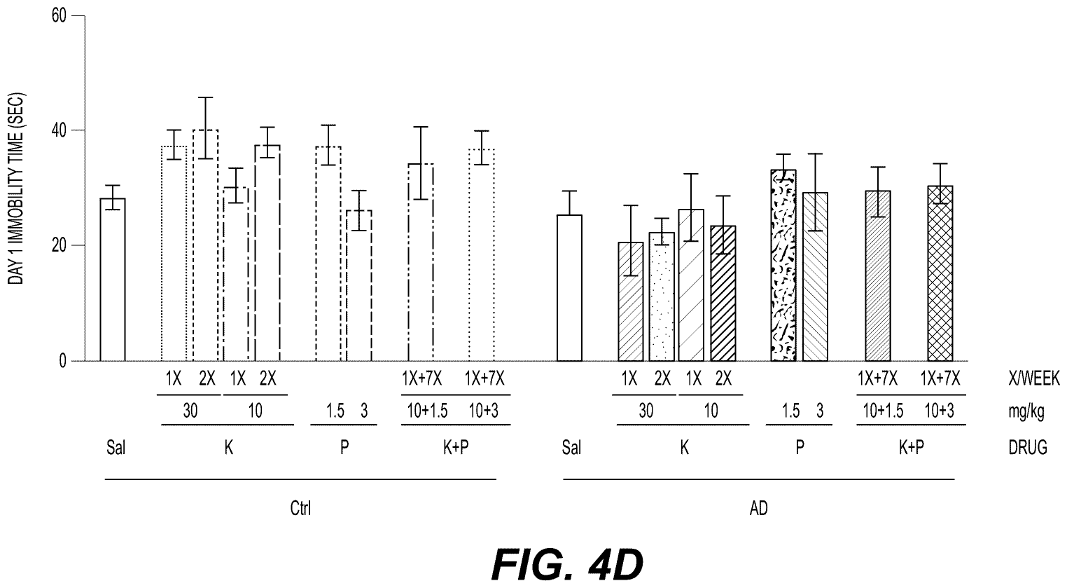

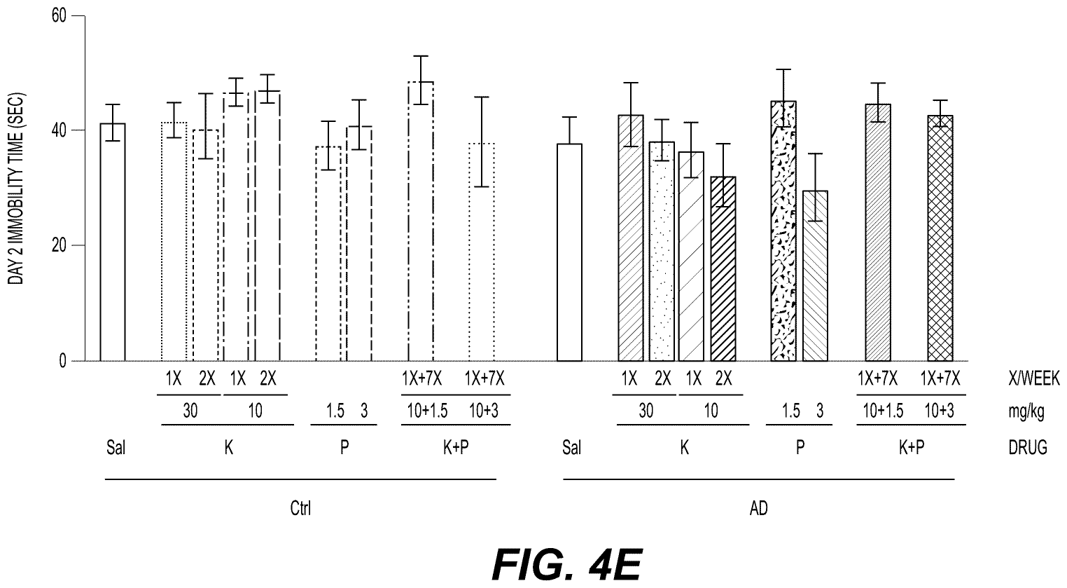

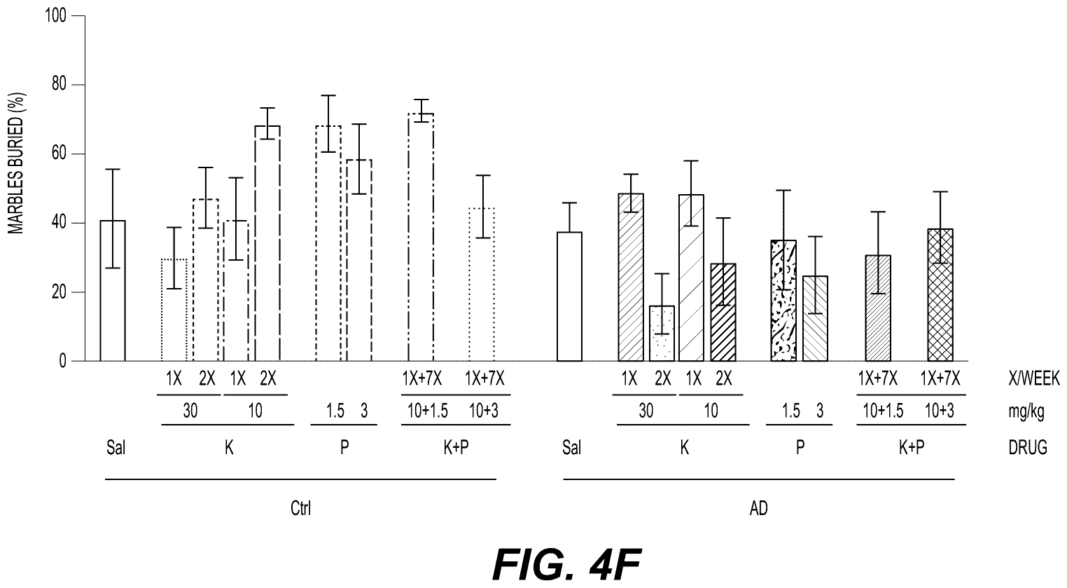

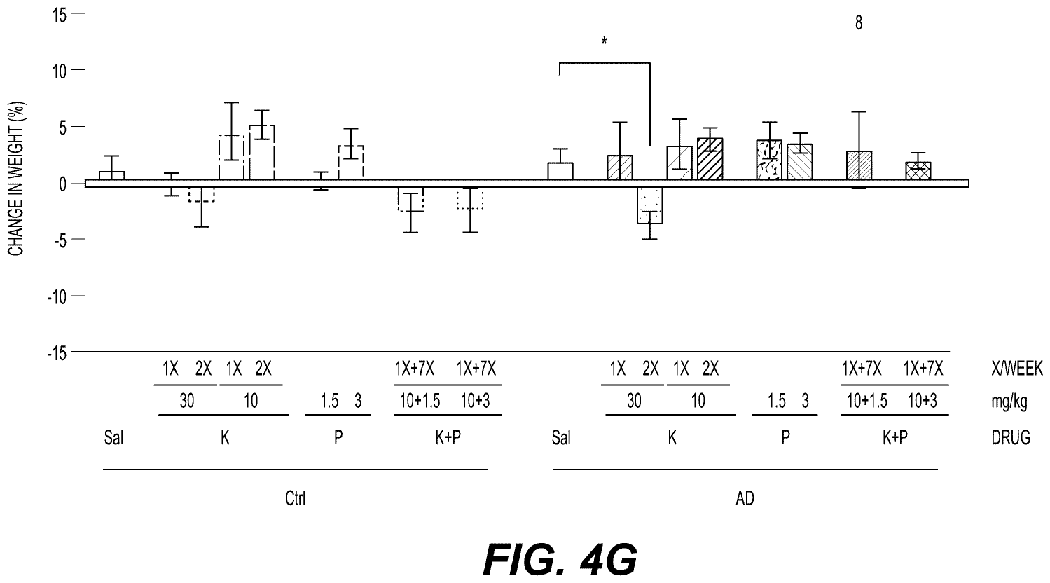

Saline-treated AD mice were impaired during memory encoding when compared with saline-treated Ctrl mice (FIG. 4B). In AD mice, 1×(R,S)-ketamine (30 mg/kg), 7× prucalopride (3 mg/kg), and 1×(R,S)-ketamine (10 mg/kg) +7×prucalopride (3 mg/kg) improved memory retrieval (FIG. 4C). All groups exhibited comparable immobility time in days 1 and 2 of the forced swim test (FIGS. 4D and 4E). All groups buried a comparable percentage of marbles (FIG. 4F) and exhibited a comparable weight loss over time (FIG. 4G).

These data suggested that combined (R,S)-ketamine and prucalopride is effective for improving memory loss in young male subjects.

### Example 8

Chronic combined (R,S)-ketamine and prucalopride administration decreases perseverative behavior in 2-month-old female APP/PS1 mice.

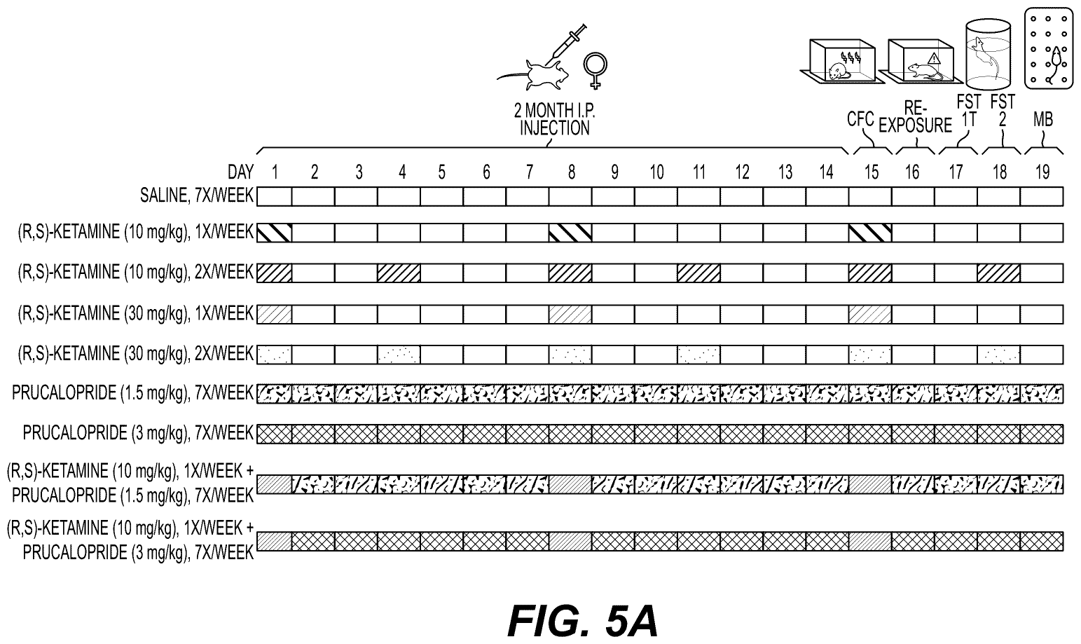

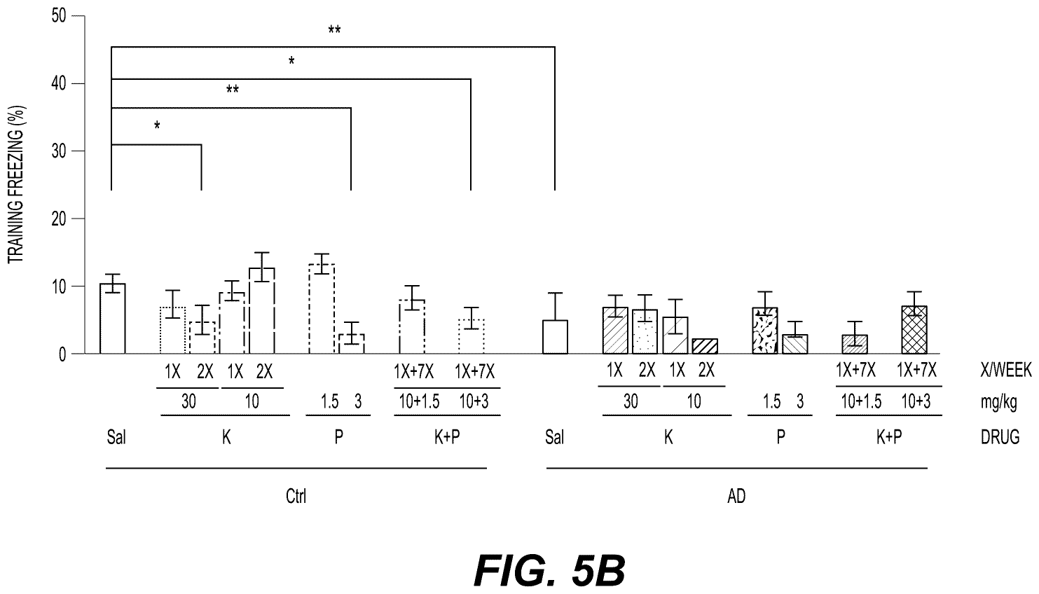

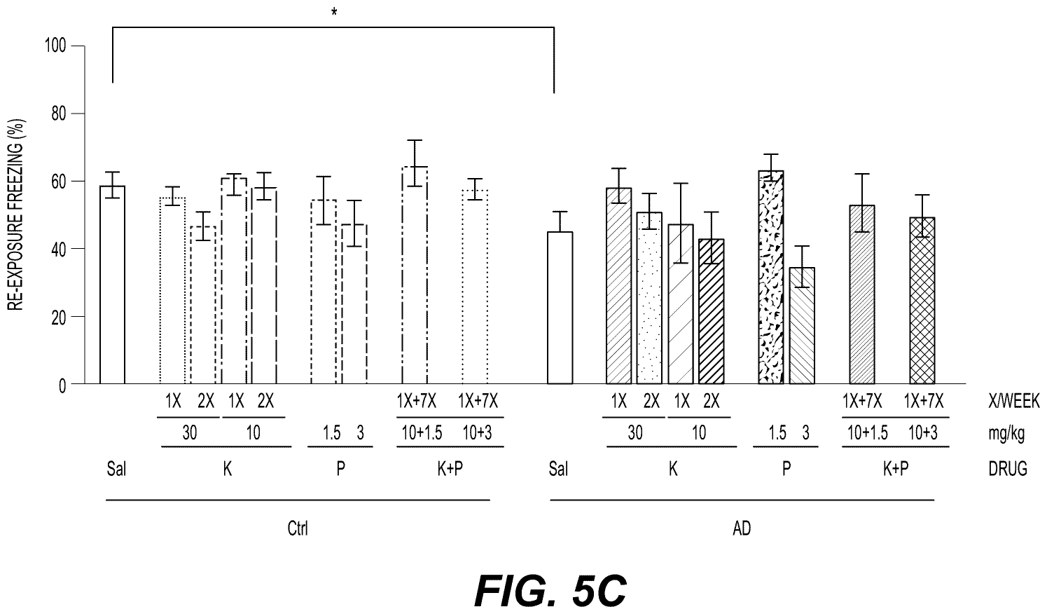

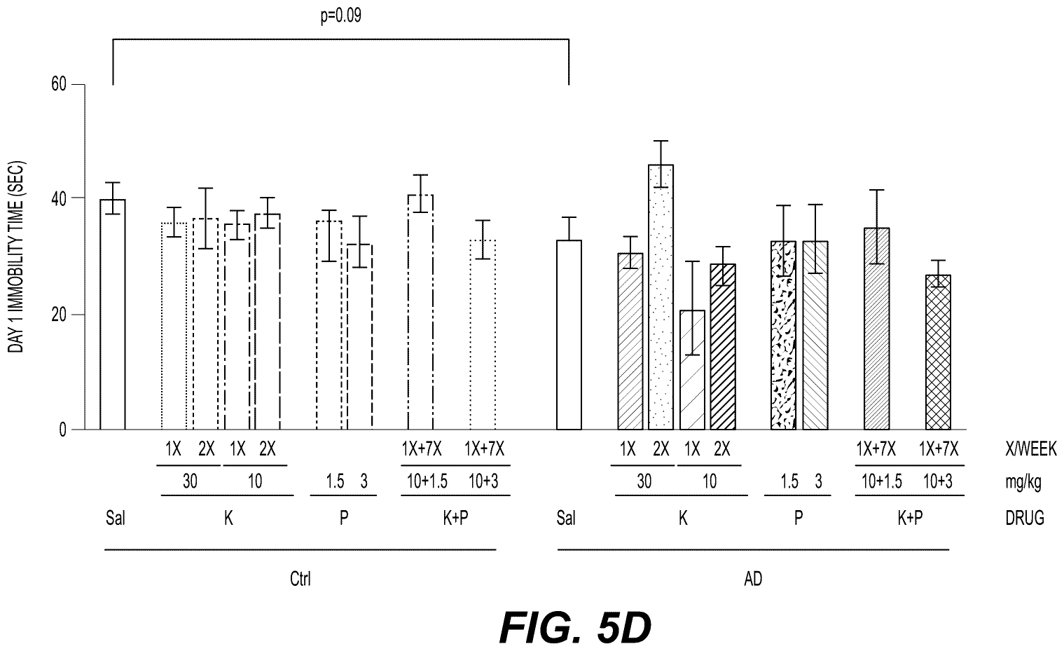

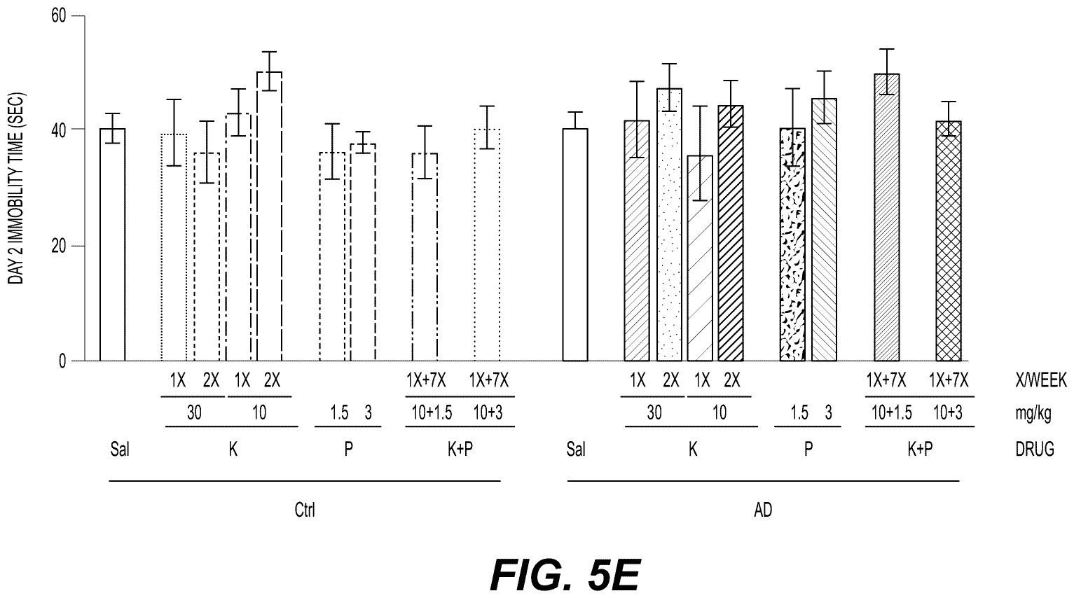

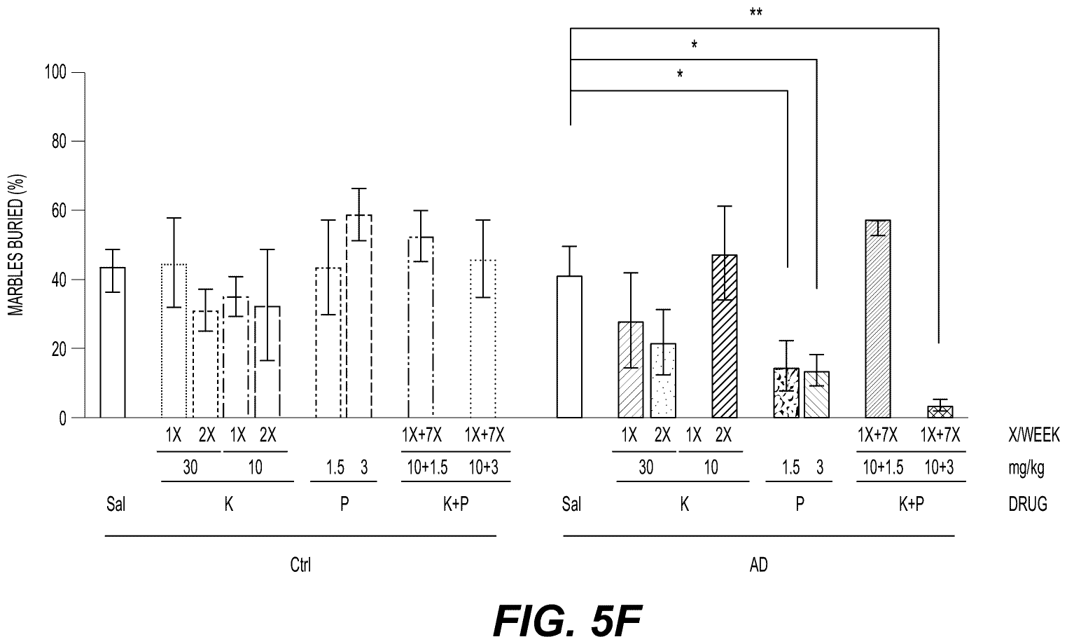

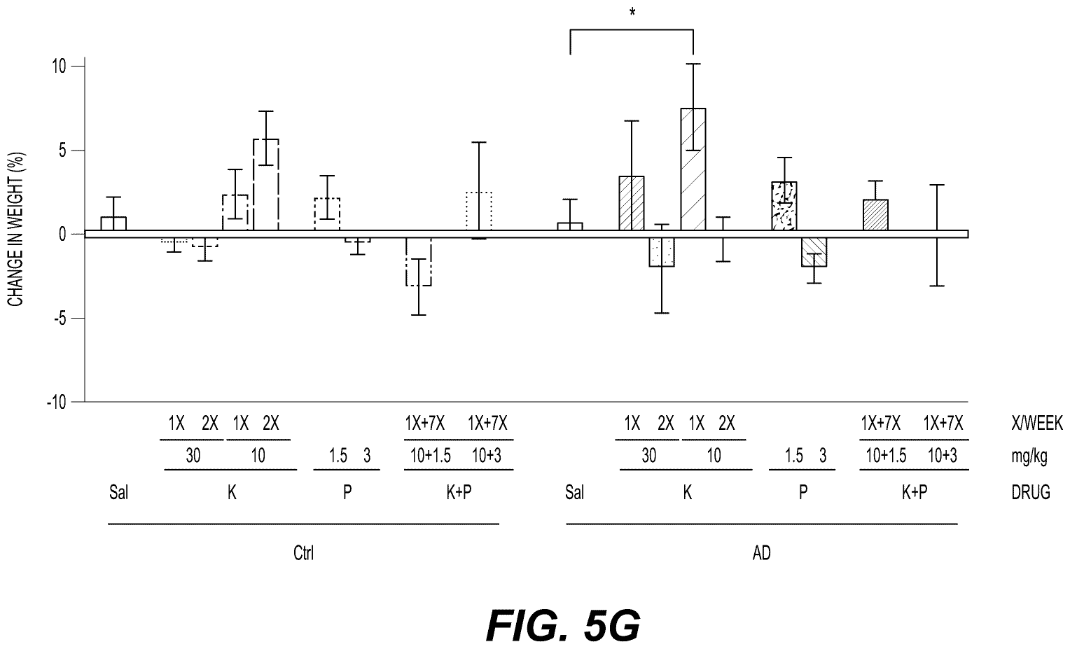

Saline-treated AD mice were impaired during memory encoding when compared with saline-treated Ctrl mice (FIG. 5B). Similarly, saline-treated AD mice were impaired during memory retrieval when compared with saline-treated Ctrl mice (FIG. 5C). All groups exhibited comparable immobility time in days 1 and 2 of the free swim test (FIGS. 5D and 5E). In the marble burying task, all groups of Ctrl mice buried a comparable percentage of marbles. In AD mice, 7×prucalopride (1.5 and 3 mg/kg), and 1×(R,S)-ketamine (10 mg/kg) +7×prucalopride (3 mg/kg) decreased the percentage of marbles buried (FIG. 5F). All groups exhibited a comparable weight loss over time (FIG. 5G).

These data suggest that combined (R,S)-ketamine and prucalopride administration is effective for improving perseverative or OCD-like behavior in young female subjects.

The disclosure of every patent, patent application, and publication cited herein is hereby incorporated herein by reference in its entirety.

While this invention has been disclosed with reference to particular embodiments, it is apparent that other embodiments and variations of this invention can be devised by others skilled in the art without departing from the true spirit and scope of the invention. The appended claims include all such embodiments and equivalent variations.

1. A method for treating Alzheimer's disease or another neurodegenerative disease and/or for improving or ameliorating symptoms associated with Alzheimer's disease in a subject in need thereof, comprising administering an effective amount of a one or more composition comprising an agonist of serotonin 4 receptor (5-HT4R), or a pharmaceutically acceptable salt, stereoisomer, tautomer, analog, derivative, or metabolite thereof, and ketamine, a ketamine analog, or a pharmaceutically acceptable salt, derivative, or metabolite thereof.

2. The method of claim 1, wherein the agonist of 5-HT4R is selected from the group consisting of: 1-(4-amino-5-chloro-2-methoxyphenyl)-3-[1(n-butyl)-4-piperidinyl]-1-propanone HCl (RS-67,333); 4-amino-5-chloro-2,3-dihydro-N-[1-3-methoxypropyl)-4-piperidinyl]-7-benzofuran carboxamide monohydrochloride (prucalopride); 4-[4-[4-Tetrahydrofuran-3-yloxy)-benzo[d]isoxazol-3-yloxymethyl]-piperidin-1-ylmethyl]-tetrahydropyran-4-ol (PF-04995274); or combinations thereof.

3. The method of claim 1, wherein the agonist of 5-HT4R is prucalopride.

4. The method of claim 1, wherein the ketamine is (R,S)-ketamine.

5. The method of claim 1, wherein the symptoms associated with Alzheimer's disease are neuropsychiatric symptoms.

6. The method of claim 5, wherein the neuropsychiatric symptoms are selected from the group consisting of: anxiety, depression, sleep disorders, appetite disorders, apathy, psychosis, perseverative behavior, agitation, aggression/irritability, euphoria, rumination, and combinations thereof.

7. The method of claim 5, wherein the neuropsychiatric symptoms are selected from the group consisting of loss of memory, impairments of language and visuospatial function, impaired learning, delusions/hallucinations, disinhibition, overall impairments in executive functions, and combinations thereof.

8. The method of claim 1, wherein the symptoms associated with Alzheimer's disease comprise cognitive symptoms.

9. The method of claim 1, wherein the symptoms associated with Alzheimer's disease comprise neuropsychiatric symptoms and cognitive symptoms.

10. The method of claim 1, wherein the one or more composition is administered orally, intravenously, intranasally, intramuscularly, topically, caudally, intrathecally, subcutaneously or via injection to the subject.

11. The method of claim 1, wherein the one or more composition is administered intravenously.

12. The method of claim 1, wherein the one or more composition is administered intranasally.

13. The method of claim 1, wherein (i) the agonist of serotonin 4 receptor (5-HT4R), or a pharmaceutically acceptable salt, stereoisomer, tautomer, analog, derivative, or metabolite thereof, and (ii) ketamine, a ketamine analog, or a pharmaceutically acceptable salt, stereoisomer, tautomer, derivative, or metabolite thereof, are administered in a single dosage form and/or concurrently.

14. The method of claim 1, wherein (i) the agonist of serotonin 4 receptor (5-HT4R), or a pharmaceutically acceptable salt, stereoisomer, tautomer, analog, derivative, or metabolite thereof, and (ii) ketamine, a ketamine analog, or a pharmaceutically acceptable salt, stereoisomer, tautomer, derivative, or metabolite thereof, are administered in separate dosage forms and/or separately.

15. The method of claim 1, wherein the subject is a human.

16. The method of claim 1, wherein (i) the agonist of serotonin 4 receptor (5-HT4R), or a pharmaceutically acceptable salt, stereoisomer, tautomer, analog, derivative, or metabolite thereof, and (ii) ketamine, a ketamine analog, or a pharmaceutically acceptable salt, stereoisomer, tautomer, derivative, or metabolite thereof, are administered at a dose ranging from about 0.01 mg/kg to about 40 mg/kg body weight of the subject.

17. The method of claim 1, wherein (i) the agonist of serotonin 4 receptor (5-HT4R), or a pharmaceutically acceptable salt, stereoisomer, tautomer, analog, derivative, or metabolite thereof, and (ii) ketamine, a ketamine analog, or a pharmaceutically acceptable salt, stereoisomer, tautomer, derivative, or metabolite thereof, are administered at a dose ranging from about 0.1 mg/kg to about 10 mg/kg body weight of the subject.

18. A method for treating Alzheimer's disease or another neurodegenerative disease and/or for improving or ameliorating symptoms associated with Alzheimer's disease in a subject in need thereof, comprising administering an effective amount of a one or more composition comprising an agonist of serotonin 4 receptor (5-HT4R), or a pharmaceutically acceptable salt, stereoisomer, tautomer, analog, derivative, or metabolite thereof, and an antagonist of the glutamate N-methyl-D-aspartate (NMDA) receptor (NMDAR).

19. A method for treating Alzheimer's disease or another neurodegenerative disease and/or for improving or ameliorating symptoms associated with Alzheimer's disease in a subject in need thereof, comprising administering an effective amount of a one or more composition comprising an agonist of serotonin 4 receptor (5-HT4R), or a pharmaceutically acceptable salt, stereoisomer, tautomer, analog, derivative, or metabolite thereof, and an agonist of the a-amino-3-hydroxy-5-methyl-4-isoxazolepropionic acid (AMPA) receptor (AMPAR).

20. The method of claim 19, wherein the agonist of AMPAR is selected from the group consisting of: AMPA, 5-fluorowillardiine, domoic acid, quisqualic acid, (2R,6R)-hydroxynorketamine, CX546, and combinations thereof.
